(19) United States
(12) Patent Application Publication
(10) Pub. No.: US 2026/0115025 A1
(43) Pub. Date: Apr. 30, 2026

(54) STENT DELIVERY DEVICE

(71) Applicant: OLYMPUS MEDICAL SYSTEMS CORP., Tokyo (JP)

(72) Inventors: Yoichi SAKURADA, Higashi-murayama-shi (JP); Kazuhiro KAN, Kunitachi-shi (JP); Takaaki HANAI, Sagamihara-shi (JP); Kanta SASAKI, Hachioji-shi (JP); Ko TAKITA, Hachioji-shi (JP); Takahira TOKIMOTO, Hachioji-shi (JP)

(21) Appl. No.: 19/008,554

(22) Filed: Jan. 2, 2025

Related U.S. Application Data
(63) Continuation of application No. PCT/JP2023/027143, filed on Jul. 25, 2023.

(51) Int. Cl. A61F 2/962; A61B 1/00

(52) U.S. Cl. CPC A61F 2/962; A61B 1/00128

(57) ABSTRACT

A stent delivery device includes an outer sheath, an inner sheath which is inserted through an inward side of the outer sheath, an outermost layer sheath through which the outer sheath is inserted on the inward side, an operation portion which has a casing coupling the inner sheath and the outermost layer sheath and is capable of operating the outer sheath and the inner sheath, and a fixing mechanism which fixes a channel of an endoscope and the outermost layer sheath.

### CROSS-REFERENCE TO RELATED APPLICATIONS

The present application is a continuation application based on PCT Patent Application No. PCT/JP2023/027143, filed on Jul. 25, 2022, the entire content of which is hereby incorporated by reference.

### FIELD

The present invention relates to a stent delivery device.

### BACKGROUND

A procedure for indwelling and expanding a stent with respect to stenosis or occlusion (which will hereinafter be referred to as “stenosis or the like”) that has occurred in the digestive tract or the like is known. A stent delivery device is used for indwelling a stent in stenosis or the like. The stent delivery devices described in the specification of U.S. Pat. No. 5,645,559 (which is referred to as Patent Document 1), the specification of U.S. Pat. No. 5,733,267 (which is referred to as Patent Document 2), and the like are inserted through a treatment tool channel of an endoscope to transport a stent to the stenosis or the like.

There is demand for stent delivery devices capable of more accurately indwelling a stent at a target location than the stent delivery devices described in Patent Document 1 and the like.

### SUMMARY

The present disclosure provides a stent delivery device in which releasing a stent at a target location is facilitated.

A stent delivery device according to a first aspect of the present invention includes an outer sheath, an inner sheath which is inserted through an inward side of the outer sheath, an outermost layer sheath through which the outer sheath is inserted on the inward side, an operation portion which has a casing coupling the inner sheath and the outermost layer sheath and is capable of operating the outer sheath and the inner sheath, and a fixing mechanism which fixes a channel of an endoscope and the outermost layer sheath.

In the stent delivery device of the present invention, releasing a stent at a target location is facilitated.

### BRIEF DESCRIPTION OF DRAWINGS

FIG. 1 is a diagram representing an endoscope system according to a first embodiment.

FIG. 2 is a diagram representing a stent delivery device of the same endoscope system.

FIG. 3 is a cross-sectional diagram of the same stent delivery device.

FIG. 4 is another cross-sectional diagram of the same stent delivery device.

FIG. 5 is a perspective diagram of an inner sheath fixing portion of the same stent delivery device.

FIG. 6 is a diagram representing an example of use of the same inner sheath fixing portion.

FIG. 7 is a diagram representing another example of use of the same inner sheath fixing portion.

FIG. 8 is a cross-sectional diagram representing a modification example of the same inner sheath fixing portion.

FIG. 9 is a cross-sectional diagram along line C1-C1 represented in FIG. 8.

FIG. 10 is a diagram representing a modification example of a lateral opening of the same stent delivery device.

FIG. 11 is another diagram representing the same modification example of the same lateral opening.

FIG. 12 is a cross-sectional diagram representing another modification example of the same inner sheath fixing portion.

FIG. 13 is a diagram representing another modification example of the same lateral opening.

FIG. 14 is a diagram representing the inner sheath fixing portion attached to the same modification example of the same lateral opening.

FIG. 15 is a cross-sectional diagram along line C2-C2 represented in FIG. 14.

FIG. 16 is a cross-sectional diagram of a stent delivery device according to a second embodiment.

FIG. 17 is a diagram representing a stretchable portion of the same stent delivery device.

FIG. 18 is a diagram representing the same stent delivery device inserted into a treatment tool channel of an endoscope.

FIG. 19 is another diagram representing the same stent delivery device being operated.

FIG. 20 is a diagram representing the same stretchable portion being folded.

FIG. 21 is another diagram representing the same stretchable portion being folded.

FIG. 22 is a diagram representing the same stent delivery device in which the same stretchable portion has been folded.

FIG. 23 is a diagram representing a stent delivery device that is another form of the second embodiment.

FIG. 24 is another diagram representing the same stent delivery device.

FIG. 25 is another diagram representing the same stent delivery device.

FIG. 26 is a diagram representing a stent delivery device that is another form of the second embodiment.

FIG. 27 is another diagram representing the same stent delivery device.

FIG. 28 is another diagram representing the same stent delivery device.

FIG. 29 is another diagram representing the same stent delivery device.

FIG. 30 is a diagram representing a stent delivery device that is another form of the second embodiment.

FIG. 31 is another diagram representing the same stent delivery device.

FIG. 32 is another diagram representing the same stent delivery device.

FIG. 33 is another diagram representing the same stent delivery device.

FIG. 34 is a diagram representing a stent delivery device that is another form of the second embodiment.

FIG. 35 is another diagram representing the same stent delivery device.

FIG. 36 is another diagram representing the same stent delivery device.

FIG. 37 is a diagram representing a stent delivery device that is another form of the second embodiment.

FIG. 38 is another diagram representing the same stent delivery device.

FIG. 39 is another diagram representing the same stent delivery device.

FIG. 40 is a diagram representing a stent delivery device that is another form of the second embodiment.

FIG. 41 is another diagram representing the same stent delivery device.

FIG. 42 is another diagram representing the same stent delivery device.

FIG. 43 is a diagram representing a stent delivery device that is another form of the second embodiment.

FIG. 44 is another diagram representing the same stent delivery device.

FIG. 45 is another diagram representing the same stent delivery device.

FIG. 46 is another diagram representing the same stent delivery device.

FIG. 47 is another diagram representing the same stent delivery device.

FIG. 48 is another diagram representing the same stent delivery device.

FIG. 49 is a diagram representing a stent delivery device that is another form of the second embodiment.

FIG. 50 is another diagram representing the same stent delivery device.

FIG. 51 is a diagram representing a stent delivery device according to a third embodiment.

FIG. 52 is a cross-sectional diagram of the same stent delivery device.

FIG. 53 is a diagram representing a stent delivery device that is another form of the third embodiment.

FIG. 54 is another diagram representing the same stent delivery device.

FIG. 55 is another diagram representing the same stent delivery device.

FIG. 56 is another diagram representing the same stent delivery device.

FIG. 57 is a diagram representing a stent delivery device that is another form of the third embodiment.

FIG. 58 is another diagram representing the same stent delivery device.

FIG. 59 is a diagram representing a stent delivery device according to a fourth embodiment.

FIG. 60 is a diagram representing a stent delivery device that is another form of the fourth embodiment.

FIG. 61 is another diagram representing the same stent delivery device.

FIG. 62 is a diagram representing a stent delivery device that is another form of the fourth embodiment.

FIG. 63 is a diagram representing a stent delivery device that is another form of the fourth embodiment.

FIG. 64 is another diagram representing the same stent delivery device.

FIG. 65 is another diagram representing the same stent delivery device.

FIG. 66 is a diagram representing a stent delivery device that is another form of the fourth embodiment.

FIG. 67 is a diagram representing a stent delivery device that is another form of the fourth embodiment.

FIG. 68 is a diagram representing a stent delivery device according to a fifth embodiment.

FIG. 69 is a cross-sectional diagram of an operation portion of the same stent delivery device.

FIG. 70 is a diagram representing a method for using the same operation portion.

FIG. 71 is a diagram representing an operation portion that is another form of the fifth embodiment.

FIG. 72 is a diagram representing an operation portion that is another form of the fifth embodiment.

FIG. 73 is another diagram representing the same operation portion.

FIG. 74 is a diagram representing an operation portion that is another form of the fifth embodiment.

FIG. 75 is another diagram representing the same operation portion.

FIG. 76 is a diagram representing an operation portion that is another form of the fifth embodiment.

FIG. 77 is a cross-sectional diagram of the same operation portion.

FIG. 78 is a perspective diagram of a rotation member of the same operation portion.

FIG. 79 is another perspective diagram of the same rotation member viewed from the opposite side.

FIG. 80 is a diagram representing a stent delivery device according to a sixth embodiment.

FIG. 81 is a cross-sectional diagram of an operation portion of the same stent delivery device.

FIG. 82 is a diagram representing a release mechanism after a stent has been released.

FIG. 83 is a diagram representing an operation portion that is another form of the sixth embodiment.

FIG. 84 is another diagram representing the same operation portion.

FIG. 85 is a diagram representing an operation portion that is another form of the sixth embodiment.

FIG. 86 is another diagram representing the same operation portion.

FIG. 87 is a diagram representing an operation portion that is another form of the sixth embodiment.

FIG. 88 is another diagram representing the same operation portion.

FIG. 89 is a diagram representing an operation portion that is another form of the sixth embodiment.

FIG. 90 is another diagram representing the same operation portion.

FIG. 91 is a diagram representing a stent delivery device according to a seventh embodiment.

FIG. 92 is a diagram representing a stent of the same stent delivery device.

FIG. 93 is another diagram representing the stent being released.

FIG. 94 is an explanatory diagram of axial collapse of the same stent.

FIG. 95 is a diagram representing a leading end portion of the same stent being released.

FIG. 96 is another diagram representing the leading end portion of the same stent being recaptured.

FIG. 97 is a graph representing a relationship between a leading end location of a high-friction member and an axial collapse reduction rate.

FIG. 98 is a diagram representing a modification example of the same high-friction member.

FIG. 99 is a diagram representing another modification example of the same high-friction member.

FIG. 100 is a diagram representing another modification example of the same high-friction member.

FIG. 101 is a diagram representing another modification example of the same high-friction member.

FIG. 102 is a diagram representing a stent delivery device according to an eighth embodiment.

### DESCRIPTION

### First Embodiment

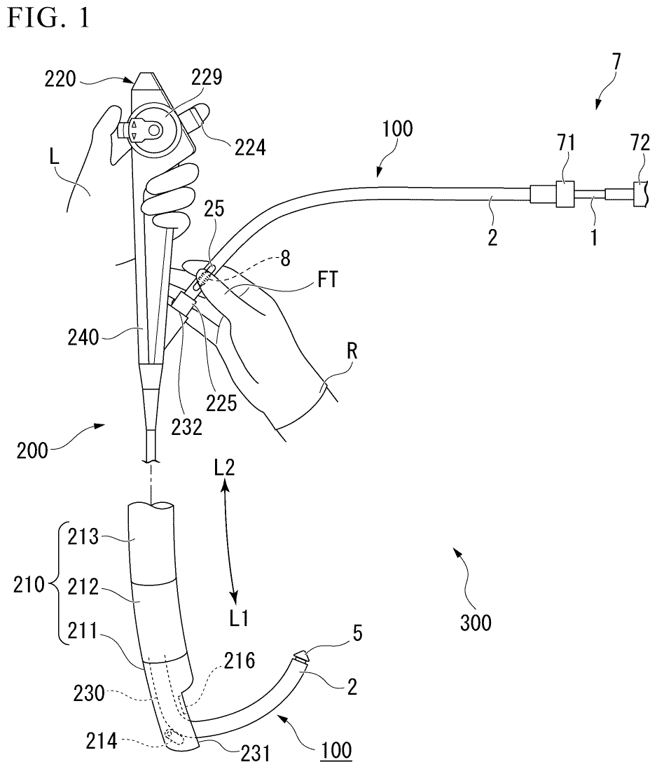

An endoscope system 300 including a stent delivery device 100 according to a first embodiment of the present invention will be described with reference to FIGS. 1 to 7.

### First Embodiment

### [Endoscope System 300]

FIG. 1 is a diagram representing the endoscope system 300.

The endoscope system 300 includes an endoscope 200 and the stent delivery device 100. The stent delivery device 100 is inserted through a treatment tool channel 230 of the endoscope 200.

### [Endoscope 200]

The endoscope 200 is a known side view-type soft endoscope, which includes a long insertion portion 210, an operation portion 220, and the treatment tool channel 230. The operation portion 220 is provided in a proximal end portion of the insertion portion 210. The treatment tool channel 230 is a channel through which a treatment tool such as the stent delivery device 100 is inserted. The endoscope 200 may be a direct view-type soft endoscope.

In the following description, the operation portion 220 side of the endoscope 200 will be referred to as a proximal end side (proximal side, proximal direction) L2. A side of the insertion portion 210 opposite to the operation portion 220 in a longitudinal axis direction L will be referred to as a leading end side (distal side, distal direction) L1 of the endoscope 200.

The insertion portion 210 has a leading end hard portion 211, a bending portion 212, and a flexible tube portion 213. The leading end hard portion 211 is provided in a leading end portion of the insertion portion 210. The bending portion 212 is attached to the proximal side of the leading end hard portion 211 and is constituted to be able to be operated in a bending manner. The flexible tube portion 213 is attached to the proximal side of the bending portion 212.

An image capturing unit 216 is provided on a side surface of the leading end hard portion 211 in a state of being exposed to the outside. The image capturing unit 216 has a light guide and a CCD.

The leading end hard portion 211 is provided with a standing base 214. A proximal end portion of the standing base 214 is rotatably supported by the leading end hard portion 211. A standing base operation wire (not illustrated) is fixed to a leading end portion of the standing base 214. The standing base operation wire extends in the proximal direction L2 through the inside of the insertion portion 210.

The bending portion 212 is constituted to be able to bend in a vertical direction and a lateral direction. In the bending portion 212, a leading end of an operation wire is fixed to the distal side of the bending portion 212. The operation wire extends to the operation portion 220 through the inside of the insertion portion 210. The vertical direction is a vertical direction in the field of view of the endoscope among orthogonal directions bending in a direction in which the insertion portion 210 intersects the axis from a state of extending straight. The lateral direction is a lateral direction in the field of view of the endoscope among orthogonal directions bending in a direction in which the insertion portion 210 intersects the axis from a state of extending straight. The bending directions of the bending portion 212 are not limited to the vertical direction and the lateral direction and can also be bending directions intersecting the axis of the insertion portion 210.

A distal end portion of the treatment tool channel 230 opens on the side surface of the leading end hard portion 211. A proximal end portion of the treatment tool channel 230 extends to the operation portion 220.

A proximal end portion of the operation portion 220 is provided with a knob 229 for operating the operation wire and a switch 224 for operating the image capturing unit 216 and the like. An operator can bend the bending portion 212 in a desired direction by operating the knob 229. The operation portion 220 need only be constituted to be able to operate the operation wire and the image capturing unit 216 and is not limited to that described above.

The operation portion 220 is provided with a forceps opening 232 which communicates with the treatment tool channel 230. The operator can insert an endoscopic treatment tool such as the stent delivery device 100 through the forceps opening 232. In order to prevent leakage of body fluid, a forceps plug 225 is attached to the forceps opening 232.

### [Stent Delivery Device 100]

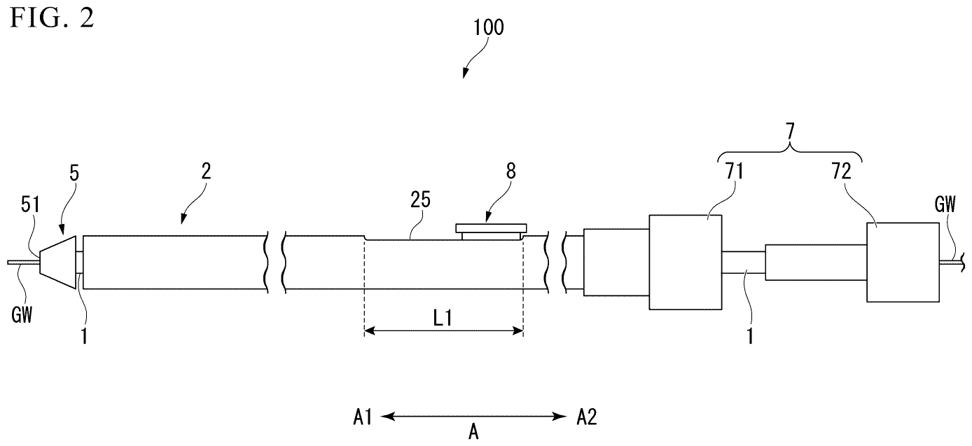

FIG. 2 is a diagram representing the stent delivery device 100.

The stent delivery device 100 is formed to be thin and long in its entirety and includes an inner sheath 1, an outer sheath 2, a tip 5, a stent 6, an operation portion 7, and an inner sheath fixing portion 8.

In the following description, in a longitudinal direction A of the stent delivery device 100, a side inserted into the body of a patient will be regarded as a leading end side (distal side, distal direction) A1 of the stent delivery device 100, and the operation portion 7 side disposed outside the body of a patient will be regarded as a proximal end side (proximal side, proximal direction) A2 of the stent delivery device 100. In addition, a direction perpendicular to the longitudinal direction A will be regarded as a radial direction R.

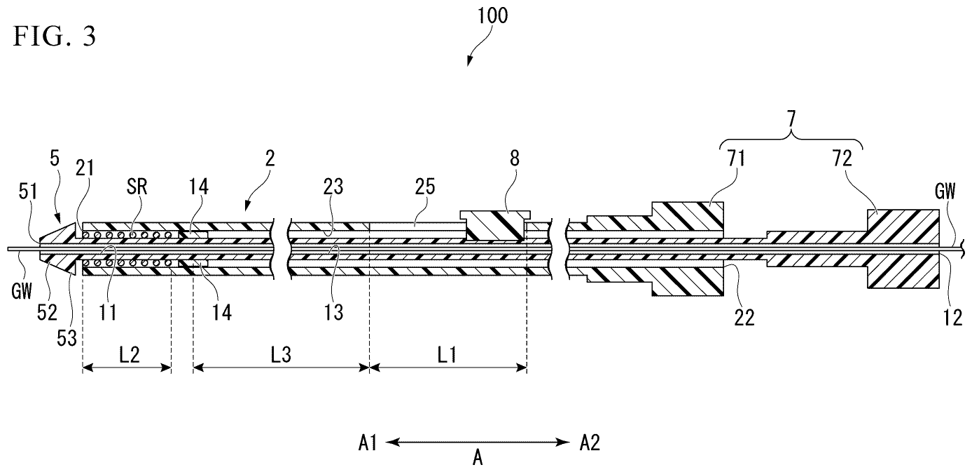

FIG. 3 is a cross-sectional diagram of the stent delivery device 100.

The outer sheath (outer tube member, second sheath) 2 is a long tubular member through which the treatment tool channel 230 of the endoscope 200 can be inserted. The outer sheath 2 is formed of a resin or the like and has flexibility. The outer sheath 2 has a first opening 21 at a leading end thereof and a second opening 22 at a proximal end thereof. The first opening 21 and the second opening 22 communicate with an internal space (lumen) 23 of the outer sheath 2. The first opening 21 and the second opening 22 are openings having substantially a circular shape through which the inner sheath 1 can be inserted.

The outer sheath 2 has a lateral opening 25 penetrated in the radial direction R. A length L1 of the lateral opening 25 in the longitudinal direction A is longer than a length L2 in the longitudinal direction A when the stent 6 is reduced in diameter. A length L3 from a stent stopper 14 to the lateral opening 25 in the longitudinal direction A is longer than the length of the treatment tool channel 230. For this reason, when the stent delivery device 100 is used by being inserted into the treatment tool channel 230 of the endoscope 200, the lateral opening 25 is disposed in a part which is not inserted into the treatment tool channel 230.

The inner sheath (inner tube member, first sheath) 1 is a long tubular member through which the internal space (guide wire lumen) 23 of the outer sheath 2 can be inserted. The inner sheath 1 is formed of a resin or the like and has flexibility. The inner sheath 1 has a leading end opening 11 at a leading end thereof and a proximal end opening 12 at a proximal end thereof. The leading end opening 11 and the proximal end opening 12 communicate with an internal space (guide wire lumen) 13 of the inner sheath 1. An outer circumferential surface of the inner sheath 1 is provided with the stent stopper 14.

The inner sheath 1 passes through the first opening 21 and the second opening 22 and is inserted through the lumen 23 of the outer sheath 2 so as to be relatively movable. The outer diameter of the inner sheath 1 inserted through the lumen 23 of the outer sheath 2 is smaller than the inner diameter of the lumen 23 of the outer sheath 2.

The tip 5 has substantially a conical shape and has a penetration hole 51 extending in the longitudinal direction A. The tip 5 has a leading end 52 with a smaller diameter size and a proximal end 53 with a larger diameter size and is connected to the inner sheath 1 on the proximal end 53 side. The diameter size of the proximal end 53 is larger than the outer diameter of the outer sheath 2. Since the penetration hole 51 communicates with the guide wire lumen 13 of the inner sheath 1 via the leading end opening 11, if a guide wire GW is inserted into the penetration hole 51 of the tip 5, the guide wire GW can enter the inside of the guide wire lumen 13 of the inner sheath 1.

The stent 6 is a tubular self-expanding stent and is formed by weaving wires. The wires forming the stent 6 are made of a super-elastic alloy having NiTi as a main material. The super-elastic alloy having NiTi as a main material is not permanently deformed when it is woven, but a woven shape is memorized by applying heat treatment in a woven state. The stent 6 may be a laser cut-type stent which is formed by cutting a metal tube with a laser.

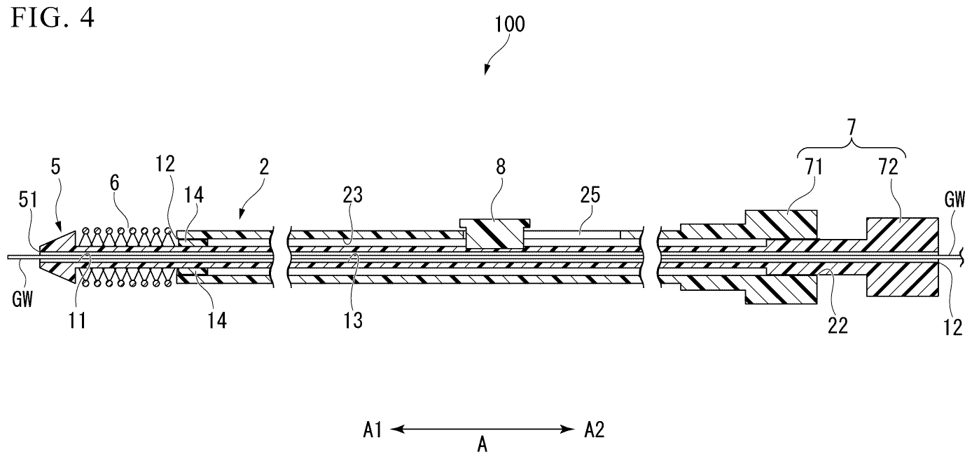

FIG. 4 is another cross-sectional diagram of the stent delivery device 100.

The stent 6 is stored in a gap between the inner sheath 1 and the outer sheath 2 in a state in which the inner sheath 1 passes through the inside thereof and is reduced in diameter. A region in which the stent 6 is stored will also be referred to as a stent storage region SR. The stent 6 is interlocked with the stent stopper 14 formed on the outer circumferential surface of the inner sheath 1. Accordingly, the location of the stent 6 is set with respect to the inner sheath 1 in a diameter-reduced state and does not move relatively in the longitudinal direction of the inner sheath 1. If the outer sheath 2 is moved to the proximal end side with respect to the inner sheath 1, the stent 6 is gradually exposed from the leading end side and expands.

The operation portion 7 is provided in a proximal portion of the stent delivery device 100. The operation portion 7 has an outer operation handle 71 and an inner operation handle 72. The operation portion 7 causes the outer sheath 2 and the inner sheath 1 to move relatively.

The outer operation handle 71 is a handle attached to a proximal end outer circumferential surface of the outer sheath 2. A user (an operator or a helper) can advance and retract the outer sheath 2 in the longitudinal direction A by operating the outer operation handle 71.

The inner operation handle 72 is a handle attached to the proximal end outer circumferential surface of the inner sheath 1. The user can advance and retract the inner sheath 1 in the longitudinal direction A by operating the inner operation handle 72.

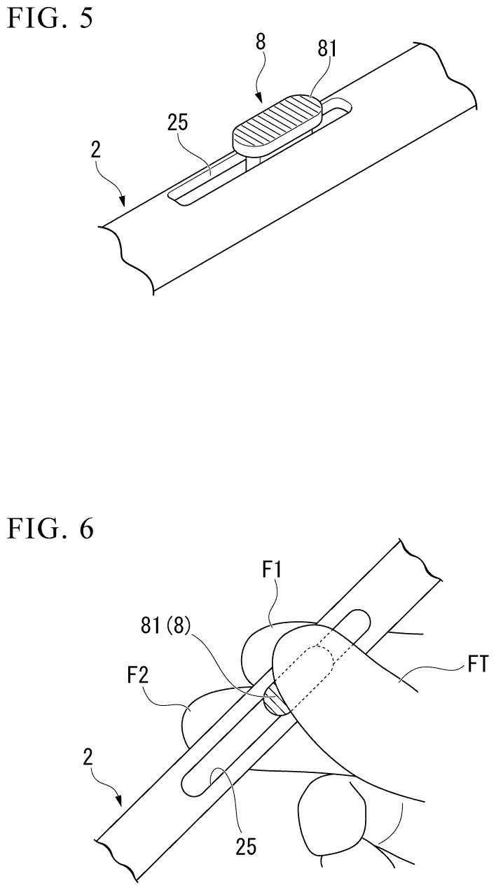

FIG. 5 is a perspective diagram of the inner sheath fixing portion 8.

The inner sheath fixing portion (engagement member, regulation mechanism) 8 is provided in an outer circumferential portion of the inner sheath 1 and protrudes in the radial direction R from the lateral opening 25 of the outer sheath 2. The inner sheath fixing portion 8 has a grip 81 on the outward side in the radial direction R.

As represented in FIG. 3, when the stent 6 is stored in the outer sheath 2, the inner sheath fixing portion 8 is disposed in the vicinity of a proximal end of the lateral opening 25. As represented in FIG. 4, when the stent 6 is exposed and released from the outer sheath 2, the inner sheath fixing portion 8 is disposed in the vicinity of a leading end of the lateral opening 25.

FIG. 6 is a diagram representing an example of use of the inner sheath fixing portion 8.

For example, the user grasps the inner sheath fixing portion 8 in a manner of pressing the grip 81 of the inner sheath fixing portion 8 with the pad of a thumb FT.

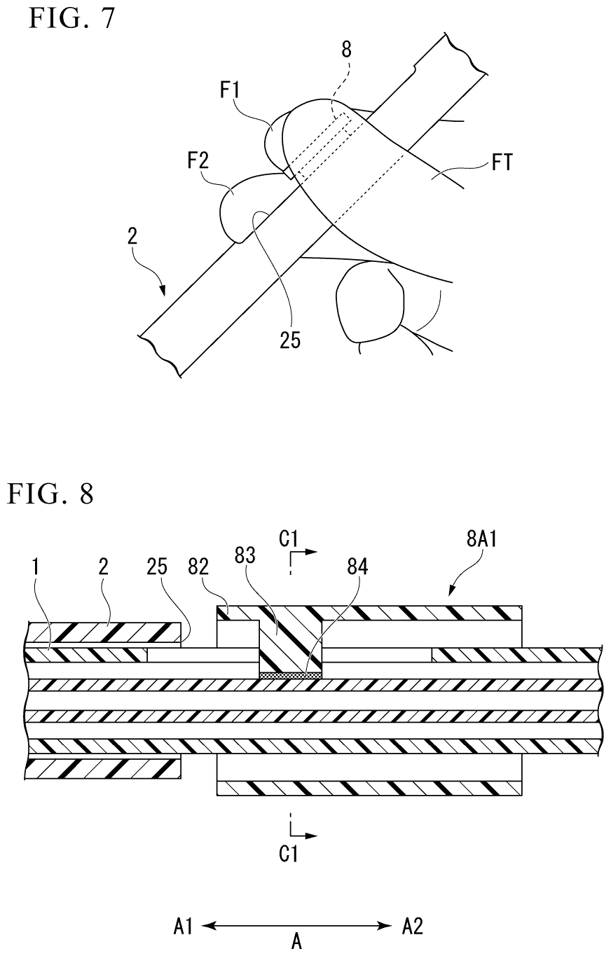

FIG. 7 is a diagram representing another example of use of the inner sheath fixing portion 8.

For example, the user grasps the inner sheath fixing portion 8 by sandwiching the inner sheath fixing portion 8 from the side with the thumb FT, an index finger F1, and a middle finger F2.

A stent indwelling method using the endoscope system 300 including the stent delivery device 100 having the foregoing constitution will be described using an exemplary procedure of indwelling the stent 6 in the bile duct as an example.

The operator inserts the insertion portion 210 of the endoscope 200 into a body cavity of a patient from a natural opening such as the mouth. At this time, as necessary, the operator bends the bending portion 212 by operating the knob 229 or the like.

After a contrast tube is inserted into the bile duct through the treatment tool channel 230 of the endoscope 200, the operator injects a contrast medium and confirms the location where stenosis has occurred in an X-ray image. Subsequently, the operator causes the guide wire GW to pass through the contrast tube, operates the guide wire GW under X-ray fluoroscopy to break through the stenosis site inside the bile duct, and moves a leading end portion of the guide wire GW to the liver side from the stenosis site (target location). Thereafter, the contrast tube is evulsed while the guide wire GW is left behind.

The operator inserts a proximal end portion of the guide wire GW protruding from the forceps plug 225 of the endoscope 200 into the penetration hole 51 of the tip 5 of the stent delivery device 100. The guide wire GW enters the guide wire lumen 13 of the inner sheath 1 from the penetration hole 51.

The operator advances the stent delivery device 100 along the guide wire GW by thrusting the stent delivery device 100 while holding the guide wire GW (insertion step). The operator causes a leading end portion of the stent delivery device 100 to protrude from the treatment tool channel 230 of the endoscope 200.

If the leading end portion of the stent delivery device 100 breaks through the stenosis site (target location), the operator determines an indwelling location of the stent 6 by advancing and retracting the stent delivery device 100. The operator may insert the stent delivery device 100 into the treatment tool channel 230 without using the guide wire GW. The length L3 from the stent stopper 14 to the lateral opening 25 in the longitudinal direction A is longer than the total length of the length of the treatment tool channel 230 and the length from a forceps standing base to the stenosis site inside the bile duct. For this reason, the inner sheath fixing portion 8 is not inserted into the treatment tool channel 230 and is disposed on the proximal end side from the forceps opening 232.

If the target location of the stent 6 is determined, as represented in FIG. 1, the operator grasps the operation portion 220 of the endoscope 200 with a left hand L and grasps the inner sheath fixing portion 8 with the thumb FT of a right hand R. The operator places the fingers other than the thumb FT of the right hand R in the vicinity of the forceps opening 232 of the endoscope 200. As a result, the inner sheath fixing portion 8 and the inner sheath 1 are fixed to the treatment tool channel 230 of the endoscope 200 (fixing step). That is, the inner sheath fixing portion 8 is a regulation member regulating movement of the inner sheath 1.

The helper retracts the outer operation handle 71 with respect to the inner operation handle 72 by operating the operation portion 7. The outer sheath 2 retracts with respect to the inner sheath 1. As a result, as represented in FIG. 4, the stent 6 is gradually exposed from the leading end side and expands.

If the stent 6 is exposed completely, the stent 6 expands in its entirety in an axial direction, and the inner diameter of the stent 6 becomes larger than the outer diameters of the inner sheath 1 and the stent stopper 14. Consequently, the interlocked state between the stent 6 and the inner sheath 1 is also canceled.

The operator grasps the inner sheath fixing portion 8 while the outer sheath 2 retracts and the stent 6 is deployed so that the location of the inner sheath 1 is fixed with respect to the treatment tool channel 230 of the endoscope 200. For this reason, the location of the inner sheath 1 is retained, and the location where the stent 6 is stored, that is, the location where the stent 6 is deployed and indwelled is unlikely to deviate from a target location.

Until the stent 6 expands completely, the stent 6 can be reduced in diameter and stored again (recaptured) between the outer sheath 2 and the inner sheath 1 by advancing the outer sheath 2 with respect to the inner sheath 1. Recapturing is useful when the indwelling location is reset or the like.

After the interlocked state between the stent 6 and the inner sheath 1 is canceled, if the user retracts the inner sheath 1, the stent 6 stays at the indwelling location, and the inner sheath 1 is evulsed from the stent 6.

If the user pulls out the stent delivery device 100 except for the stent 6 from the body, the procedure of indwelling the stent 6 ends. Thereafter, the contrast tube may be introduced along the guide wire GW, and the patency state of stenosis may be confirmed using the contrast medium.

According to the stent delivery device 100 of the present embodiment, the location of the inner sheath 1 with respect to the treatment tool channel 230 is fixed, and therefore the location where the stent 6 is stored is unlikely to deviate from a target location.

In stent delivery devices in the related art, when the outer sheath is retracted to the proximal end side, the outer sheath and the inner sheath disposed on the hand side become straight from the forceps plug 225, which may cause deviation of the inner sheath to the leading end side, for example. According to the stent delivery device 100 of the present embodiment, such deviation of the inner sheath can be curbed, and releasing at a target location is facilitated.

Hereinabove, the first embodiment of the present invention has been described in detail with reference to the drawings, but the specific constitutions are not limited to this embodiment, and design change and the like within a range not departing from the gist of the present invention are also included. In addition, the constituent elements described in the foregoing embodiment and the following modification examples can be suitably constituted in a combination.

### Modification Example 1-1

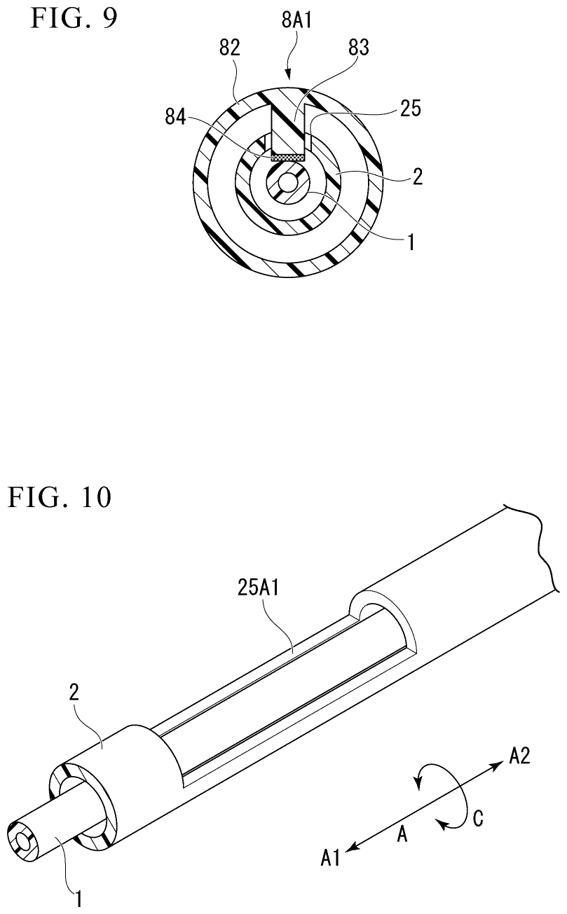

FIG. 8 is a cross-sectional diagram representing an inner sheath fixing portion 8A1 that is a modification example of the inner sheath fixing portion 8. FIG. 9 is a cross-sectional diagram along line C1-C1 represented in FIG. 8. The inner sheath fixing portion 8A1 has a falling prevention sheath 82 and a push bar 83. The falling prevention sheath 82 can slide on an outer circumferential surface of the outer sheath 2 in the longitudinal direction A. The push bar 83 is attached to an inner circumferential surface of the falling prevention sheath 82 and is inserted through the lateral opening 25. The push bar 83 can come into contact with the inner sheath 1, and a pressing portion 84 is formed in the contact part. The pressing portion 84 is formed of a material having a high frictional coefficient (for example, silicone rubber). The operator brings the pressing portion 84 into contact with the inner sheath 1 by thrusting the inner sheath fixing portion 8A1, thereby fixing the inner sheath 1. Since the falling prevention sheath 82 can move along the outer sheath 2, it is easy for the operator to press the inner sheath fixing portion 8A1.

### Modification Example 1-2

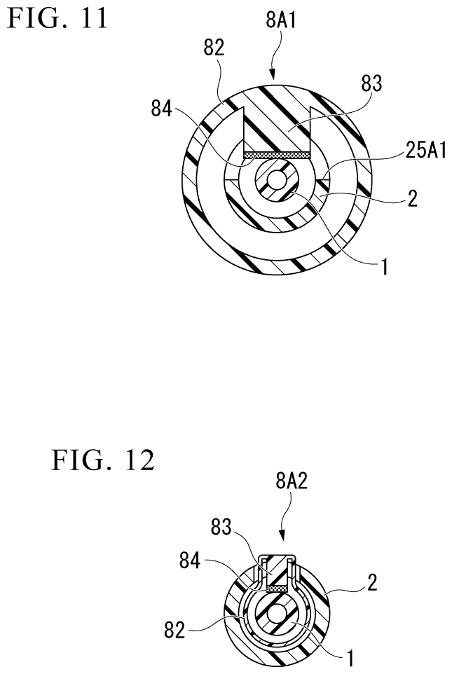

FIGS. 10 and 11 are diagrams representing a lateral opening 25A1 that is a modification example of the lateral opening 25. The lateral opening 25A1 is an opening formed by removing approximately half of the outer sheath 2 in a circumferential direction C. The opening area of the lateral opening 25A1 is larger than the opening area of the lateral opening 25. By increasing the widths of the push bar 83 and the pressing portion 84 of the inner sheath fixing portion 8A1 attached to the lateral opening 25A1, the inner sheath fixing portion 8A1 can favorably fix the inner sheath 1.

### Modification Example 1-3

FIG. 12 is a cross-sectional diagram representing an inner sheath fixing portion 8A2 that is another modification example of the inner sheath fixing portion 8. The inner sheath fixing portion 8A2 differs from the inner sheath fixing portion 8A1 in that the falling prevention sheath 82 is disposed between the outer sheath 2 and the inner sheath 1. Since the inner sheath fixing portion 8A2 is disposed along the inner sheath 1, the pressing portion 84 reliably comes into contact with the inner sheath 1 so that the inner sheath 1 can be fixed more reliably.

### Modification Example 1-4

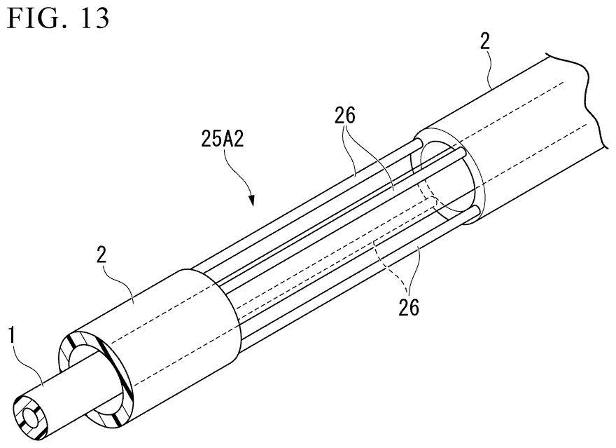

FIG. 13 is a diagram representing lateral openings 25A2 that are another modification example of the lateral opening 25. The lateral openings 25A2 are gaps between connection wires 26 coupling the divided outer sheath 2.

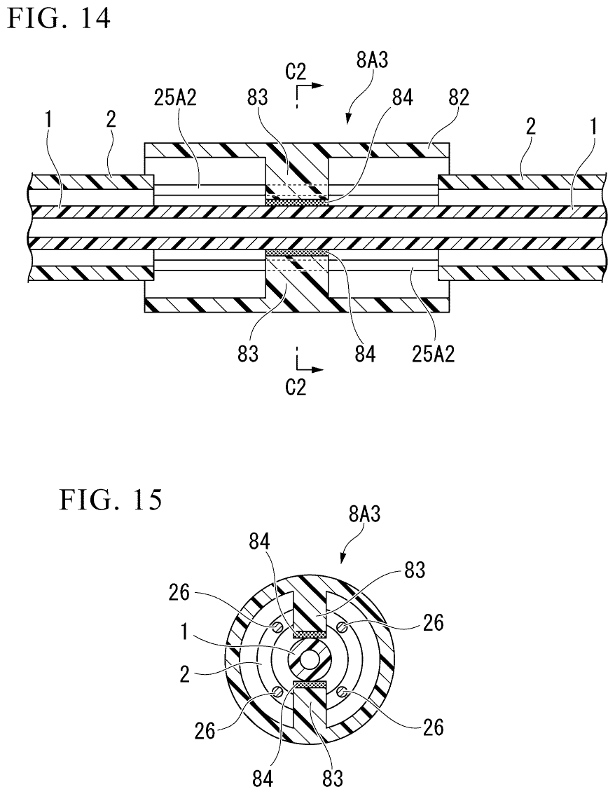

FIG. 14 is a diagram representing an inner sheath fixing portion 8A3 attached to the lateral openings 25A2. FIG. 15 is a cross-sectional diagram along line C2-C2 represented in FIG. 14. In the inner sheath fixing portion 8A3, the push bar 83 and the pressing portion 84 are provided on both sides sandwiching the inner sheath 1 therebetween. The inner sheath fixing portion 8A3 can sandwich the inner sheath 1 from both sides so that the inner sheath 1 can be fixed more reliably.

### Second Embodiment

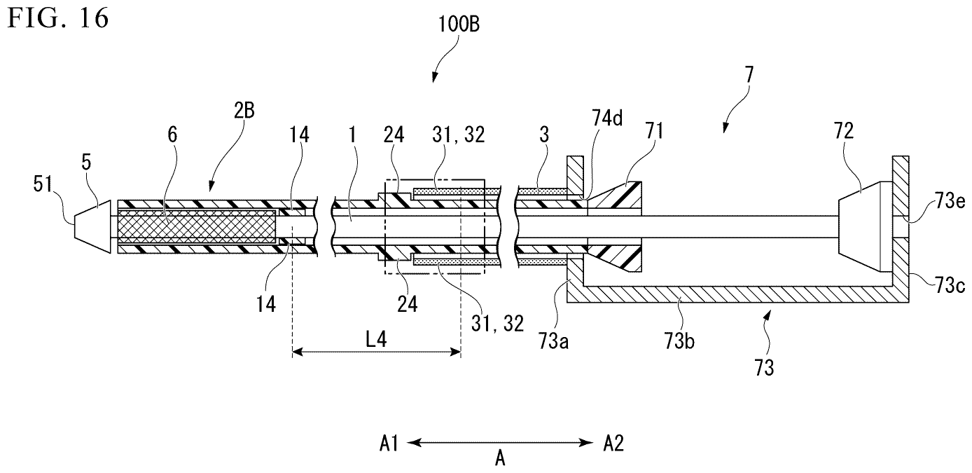

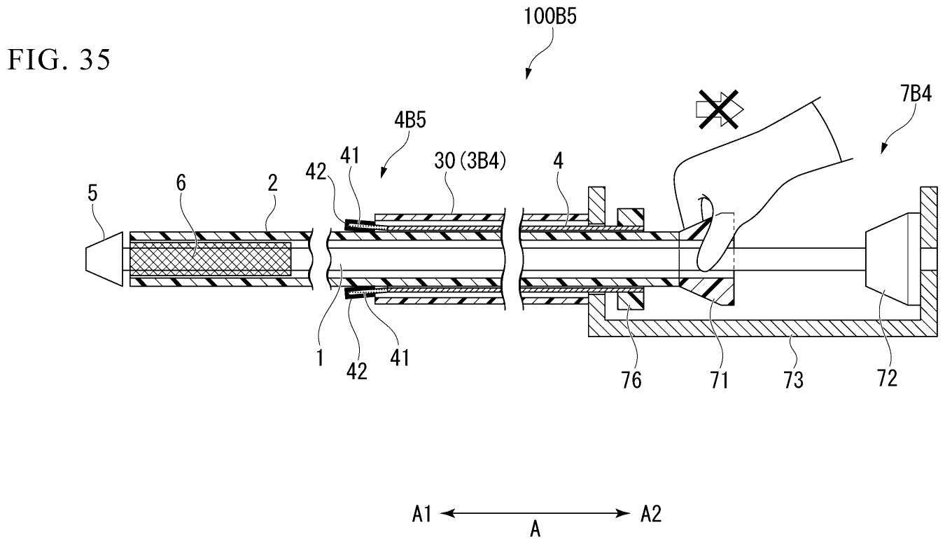

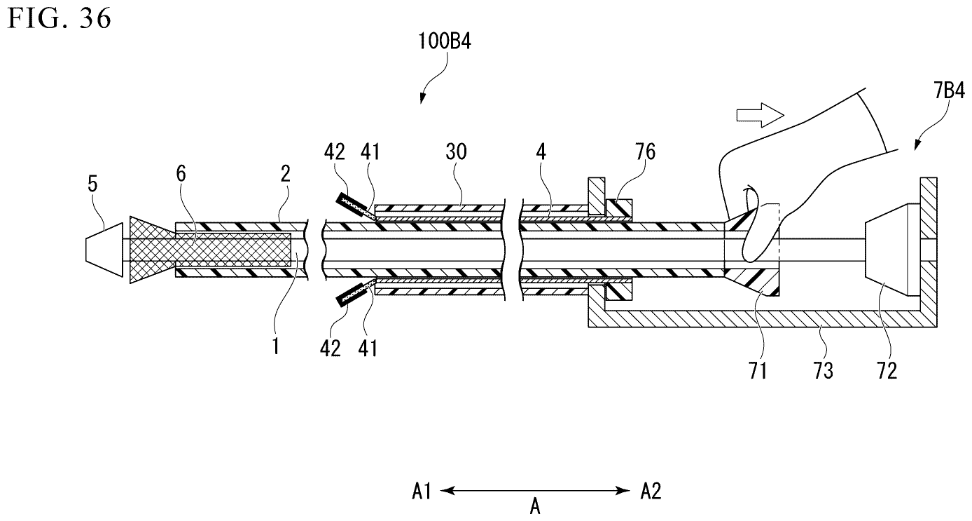

A second embodiment of the present invention will be described with reference to FIGS. 16 to 50. In the following description, the same reference signs are applied to common constituents which have already been described, and duplicate description will be omitted.

FIG. 16 is a cross-sectional diagram of a stent delivery device 100B according to the present embodiment.

Similar to the stent delivery device 100 of the first embodiment, the stent delivery device 100B is inserted through the treatment tool channel 230 of the endoscope 200.

The stent delivery device 100B is formed to be thin and long in its entirety and includes the inner sheath (first sheath) 1, an outer sheath (second sheath) 2B, an outermost layer sheath (third sheath) 3, the tip 5, the stent 6, and an operation portion 7B. The stent delivery device 100B has a triple sheath structure in which the inner sheath 1, the outer sheath 2B, and the outermost layer sheath 3 overlap.

The outer sheath (second sheath) 2B is the same as the outer sheath 2 of the first embodiment except that a cylindrical portion 24 is provided in the outer circumferential portion. The cylindrical portion 24 has a larger outer diameter in the radial direction R than other parts of the outer sheath 2. The cylindrical portion 24 is provided closer to the leading end side than a leading end portion of the outermost layer sheath 3.

The operation portion 7B has the outer operation handle 71, the inner operation handle 72, and a casing 73.

The casing 73 is a case surrounding the outer operation handle 71 and the inner operation handle 72. The casing 73 has a casing leading end portion 73a, a casing main body 73b, and a casing proximal end portion 73c. The casing leading end portion 73a, the casing main body 73b, and the casing proximal end portion 73c are arranged from the leading end side to the proximal end side in the longitudinal direction A.

The casing leading end portion 73a is provided at a leading end of the casing main body 73b. The casing leading end portion 73a is disposed on the leading end side from the outer operation handle 71. The casing leading end portion 73a is provided with a leading end penetration hole 74d through which the outer sheath 2B is inserted to be able to advance and retract.

The casing main body 73b is a member coupling the casing leading end portion 73a and the casing proximal end portion 73c and extends in the longitudinal direction A. It is desirable that the casing main body 73b have a grip or the like to be grasped by the user.

The casing proximal end portion 73c is attached to the proximal ends of the inner sheath 1 and the inner operation handle 72. The casing proximal end portion 73c has a proximal end penetration hole 73e which communicates with the guide wire lumen 13 of the inner sheath 1.

The outermost layer sheath (third sheath) 3 is a sheath disposed on the outward side of the outer sheath 2B in the radial direction R. A proximal end of the outermost layer sheath 3 is fixed to the casing leading end portion 73a. That is, the outermost layer sheath 3 is coupled to the inner sheath 1 via the casing 73.

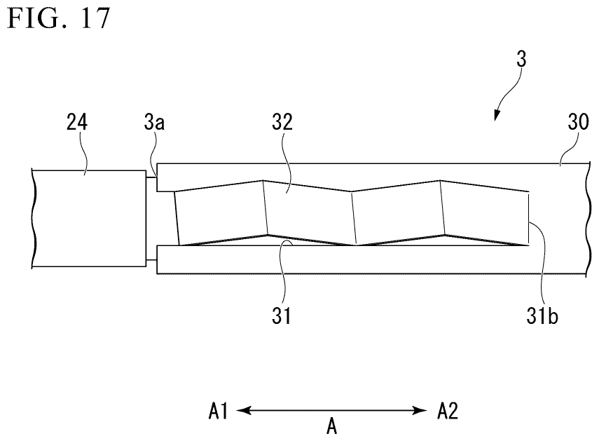

FIG. 17 is a diagram representing a stretchable portion 32.

The outermost layer sheath 3 has an outermost layer sheath main body 30 which is a long tubular member, a cutout portion 31, and the stretchable portion 32. The cutout portion 31 and the stretchable portion 32 are provided in a leading end portion of the outermost layer sheath main body 30. The cutout portion 31 and the stretchable portion 32 are provided on both sides sandwiching the center axis of the outermost layer sheath 3 in the longitudinal direction A therebetween.

The cutout portion 31 is a slit extending in the longitudinal direction A from a leading end 3a of the outermost layer sheath 3 and penetrates the outermost layer sheath 3 in the radial direction R. The length of the cutout portion 31 in the longitudinal direction A is 150 mm to 200 mm, for example.

For example, the stretchable portion 32 is a member which stretches and shrinks by being folded as in a bellows shape. It is desirable that the stretchable portion 32 be formed of a material having a high frictional coefficient. A proximal end of the stretchable portion 32 is connected to a proximal end 31b of the cutout portion 31. The stretchable portion 32 covers almost the entire area of the cutout portion 31 in a stretched state. The stretchable portion 32 is creased so as to be folded alternately when it shrinks.

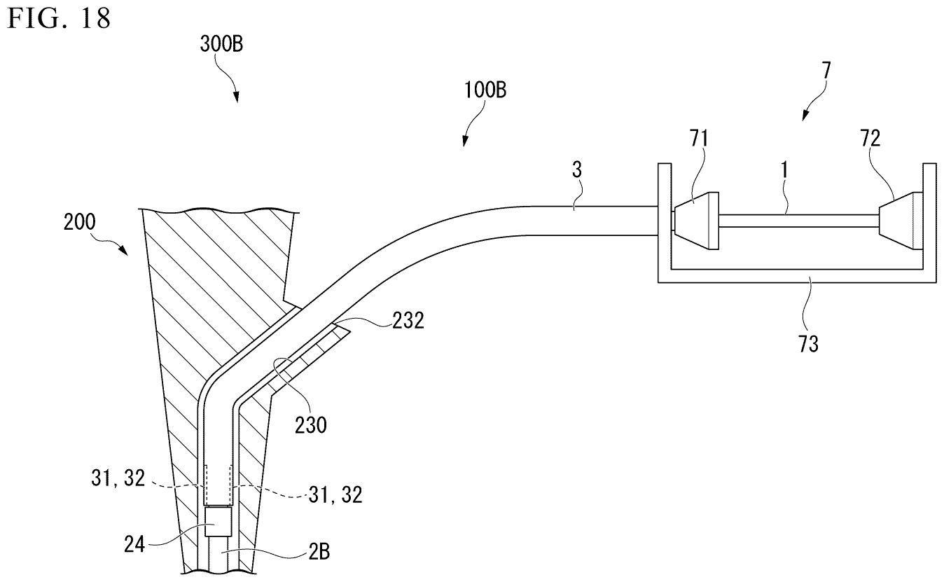

FIG. 18 is a diagram representing the stent delivery device 100B inserted into the treatment tool channel 230. The outermost layer sheath 3 does not extend to a leading end of the outer sheath 2B. A length L4 from the stent stopper 14 to the proximal end 31b of the cutout portion 31 of the outermost layer sheath 3 in the longitudinal direction A is longer than the length of the treatment tool channel 230. For this reason, when the stent delivery device 100B is used by being inserted into the treatment tool channel 230 of the endoscope 200, the stretchable portion 32 is disposed in a part which is inserted into the treatment tool channel 230. The length L4 is 1.5 m to 2 m, for example.

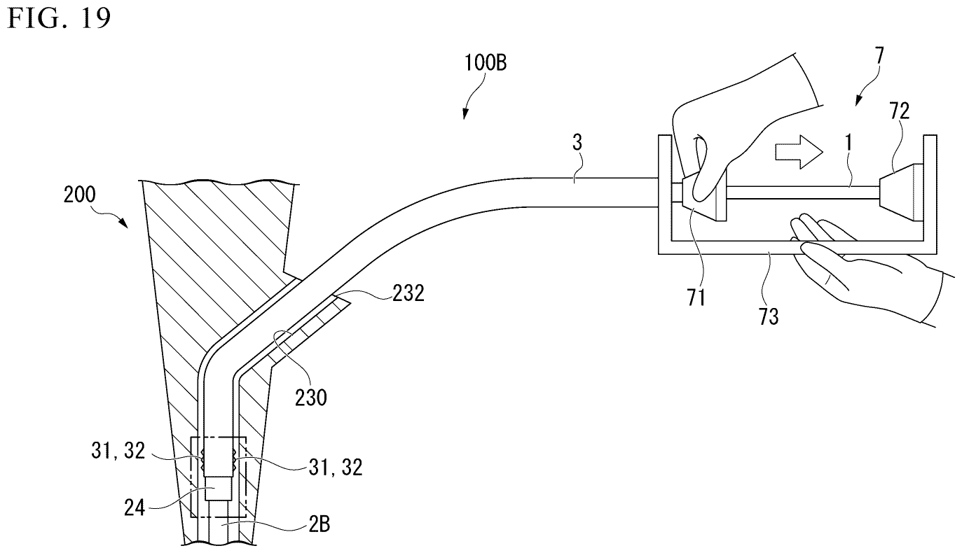

FIG. 19 is another diagram representing the stent delivery device 100B being operated.

The helper retracts the outer operation handle 71 with the right hand R by operating the operation portion 7, for example, in a state of holding the casing 73 with the left hand L. The outer sheath 2B retracts with respect to the inner sheath 1.

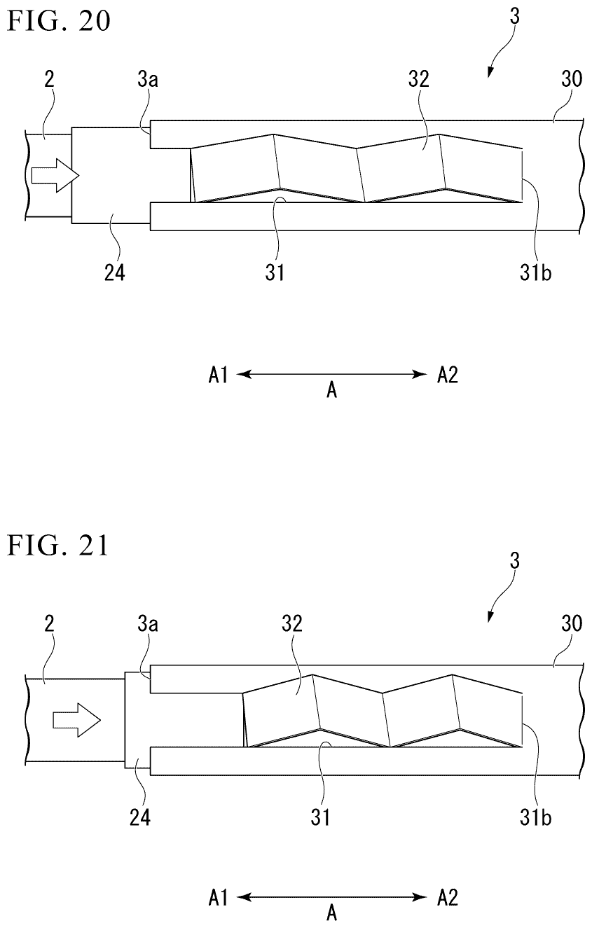

FIGS. 20 and 21 are diagrams representing the stretchable portion 32 being folded.

The outer sheath 2B retracts with respect to the inner sheath 1. As in FIGS. 20 and 21, the stretchable portion 32 is folded when it comes into contact with the cylindrical portion 24 retracting. When the stretchable portion 32 is folded, the length in the longitudinal direction A is gradually shortened.

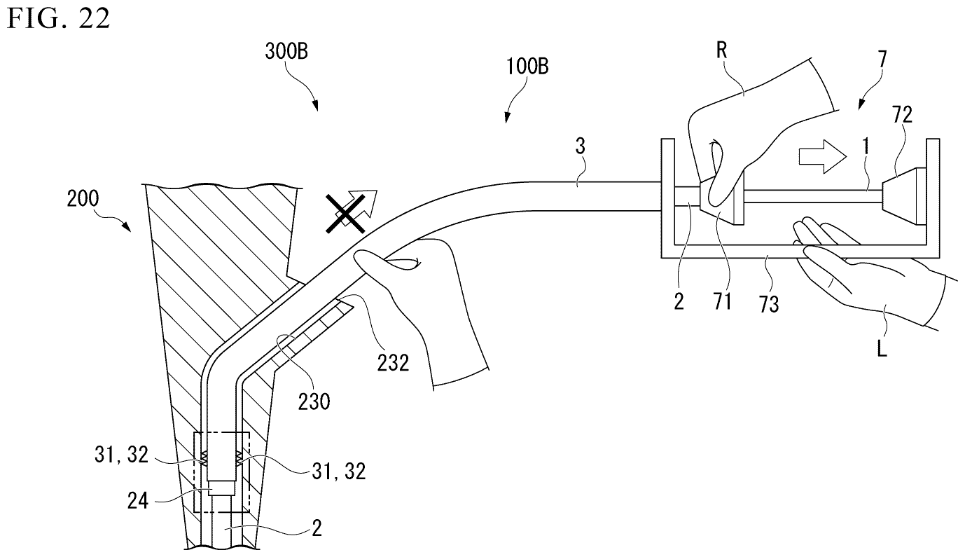

FIG. 22 is a diagram representing the stent delivery device 100B in which the stretchable portion 32 has been folded. When the stretchable portion 32 is folded, it is sandwiched between the treatment tool channel 230 and the outer sheath 2B. As a result, a frictional force generated between the outermost layer sheath 3 and the treatment tool channel 230 increases, and the outermost layer sheath 3 is fixed to the treatment tool channel 230. The stretchable portion 32 is a fixing mechanism for fixing the treatment tool channel 230 and the outermost layer sheath 3. Since the inner sheath 1 is coupled to the outermost layer sheath 3 via the casing 73, the inner sheath 1 is also fixed to the treatment tool channel 230. That is, the stretchable portion (fixing mechanism) 32 is a regulation member regulating movement of the inner sheath 1.

The helper further retracts the outer operation handle 71 with respect to the inner operation handle 72. The outer sheath 2 retracts with respect to the inner sheath 1. As a result, the stent 6 is gradually exposed from the leading end side and expands. The inner sheath 1 is fixed to the treatment tool channel 230. For this reason, the location of the inner sheath 1 is retained, and the location where the stent 6 is stored is unlikely to deviate from a target location.

In stent delivery devices in the related art, when the outer sheath is retracted to the proximal end side, the inner sheath may deviate to the leading end side. At this time, an operator grasping the stent delivery device performs an operation of drawing the inner sheath into the proximal end side (which will hereinafter be referred to as “a cooperative operation”) so as to correct deviation of the inner sheath to the leading end side. According to the stent delivery device 100B of the present embodiment, such a cooperative operation is not necessary. In addition, even if the operator attempts to perform such a cooperative operation, since the outermost layer sheath 3 is fixed to the treatment tool channel 230, the operator cannot perform the cooperative operation. For this reason, problems that may be caused by performing a cooperative operation, which is originally unnecessary, will not occur.

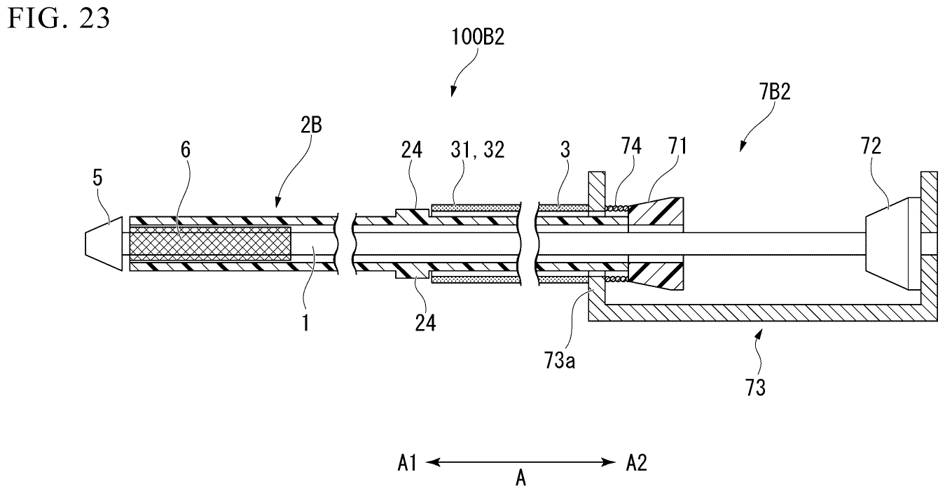

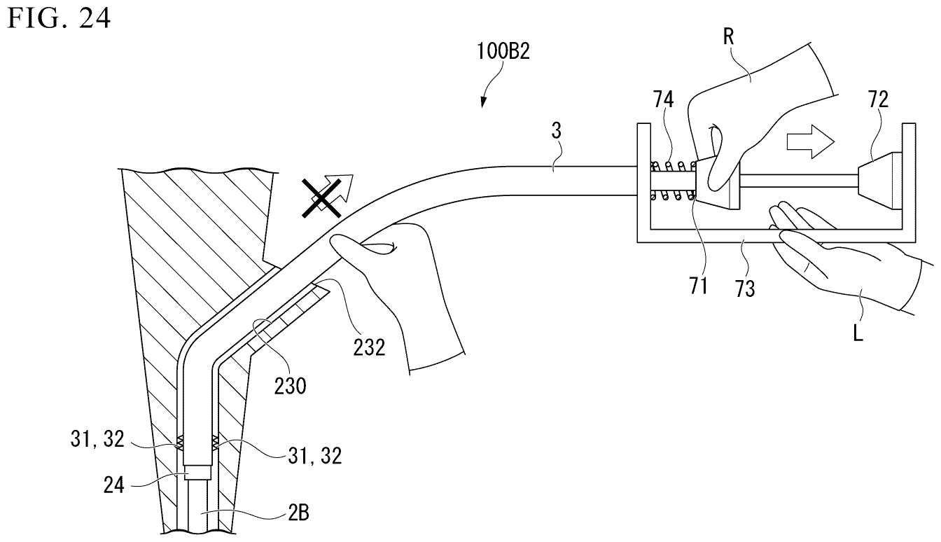

FIGS. 23 to 25 are diagrams representing a stent delivery device 100B2 that is another form of the present embodiment. The stent delivery device 100B2 includes the inner sheath 1, the outer sheath 2B, the outermost layer sheath 3, the tip 5, the stent 6, and an operation portion 7B2.

The operation portion 7B2 has the outer operation handle 71, the inner operation handle 72, the casing 73, and an elastic member 74. The elastic member 74 is a spring or the like. The elastic member 74 couples the casing leading end portion 73a of the casing 73 and the outer operation handle 71 to each other. The elastic member 74 is biased in a direction in which the casing leading end portion 73a and the outer operation handle 71 approach each other.

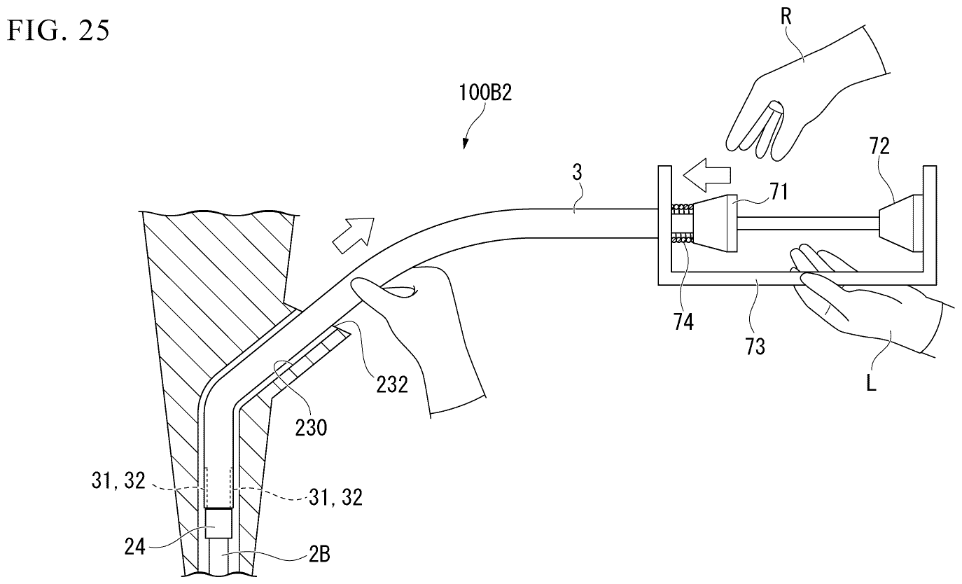

As represented in FIGS. 24 and 25, if the helper releases the outer operation handle 71 which has been retracted with the right hand R, the outer operation handle 71 advances toward the casing leading end portion 73a due to a biasing force of the elastic member 74. When the cylindrical portion 24 advances, the stretchable portion 32 stretches. As a result, the fixed state of the outermost layer sheath 3 with respect to the treatment tool channel 230 is canceled.

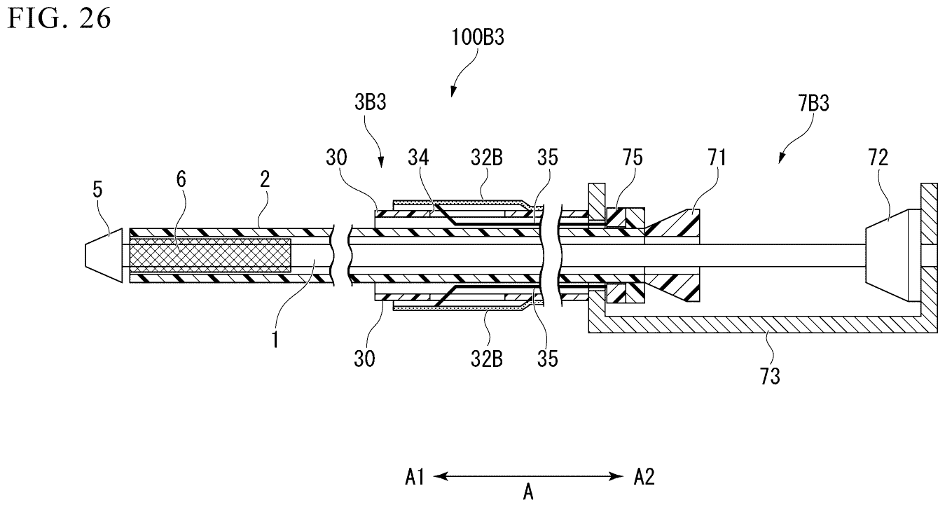

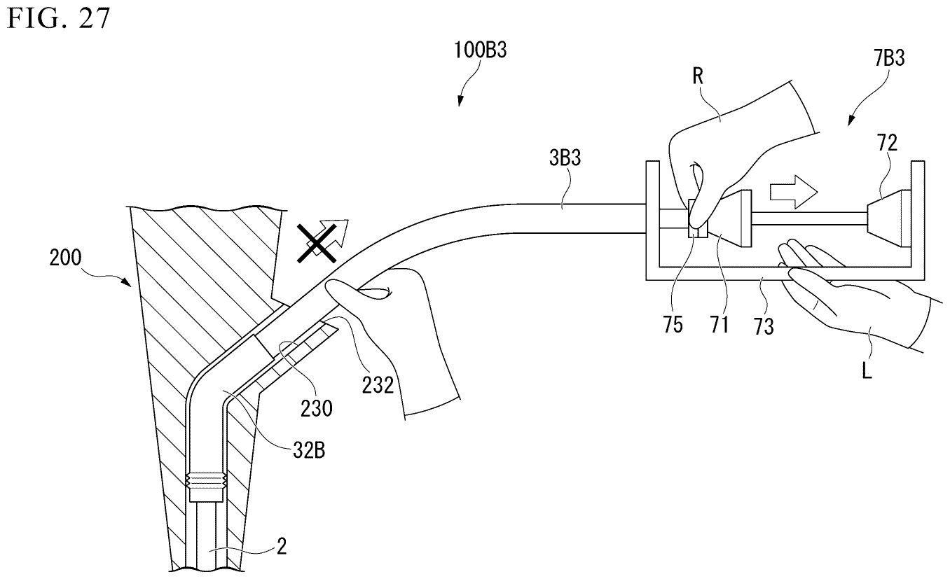

FIGS. 26 to 29 are diagrams representing a stent delivery device 100B3 that is another form of the present embodiment. The stent delivery device 100B3 includes the inner sheath 1, the outer sheath 2, an outermost layer sheath 3B3, the tip 5, the stent 6, and an operation portion 7B3.

The outermost layer sheath 3B3 has the outermost layer sheath main body 30 provided with a slit 34 lying in the longitudinal direction A, a stretchable portion 32B disposed on the outward side of the outermost layer sheath main body 30, and a wire 35 coupled to the stretchable portion 32B. The wire 35 is inserted through the slit 34.

The operation portion 7B3 has the outer operation handle 71, the inner operation handle 72, the casing 73, and a wire operation handle 75. The wire operation handle 75 is attached to the outer sheath 2 so as to be slidable with respect to the outer sheath 2. The wire 35 is attached to the wire operation handle 75.

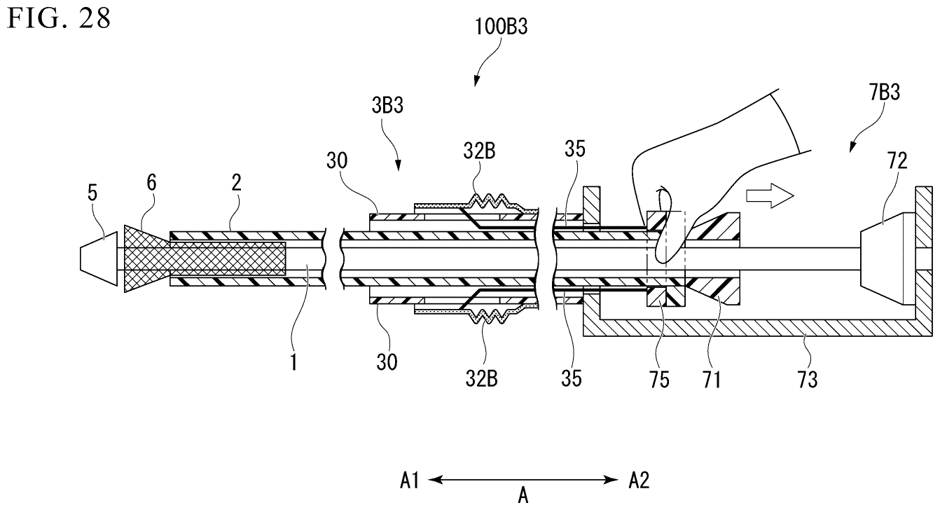

As represented in FIGS. 27 and 28, if the helper retracts the wire operation handle 75 with the right hand R, the outer operation handle 71 also retracts. As a result, the outer sheath 2 retracts, and the wire 35 also retracts so that the stretchable portion 32B is pulled. The stretchable portion 32B pulled to the proximal end side is folded and thereby sandwiched between the treatment tool channel 230 and the outermost layer sheath main body 30. As a result, the outermost layer sheath 3B3 is fixed to the treatment tool channel 230, and the inner sheath 1 is fixed to the treatment tool channel 230. By performing one simple operation of retracting the wire operation handle 75, the outer sheath 2 and the stretchable portion 32B can be pulled.

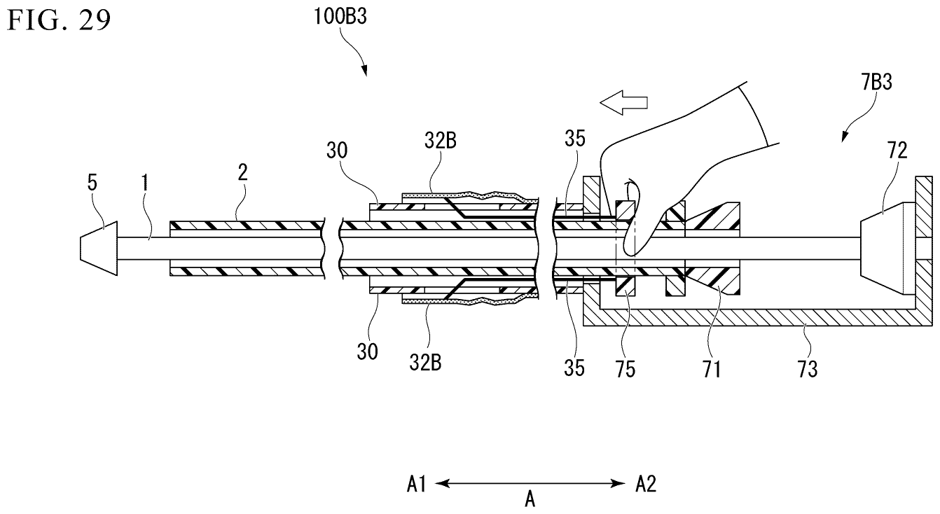

As represented in FIG. 29, if the helper advances the wire operation handle 75, the stretchable portion 32B stretches. As a result, the fixing state of the outermost layer sheath 3B3 with respect to the treatment tool channel 230 is canceled.

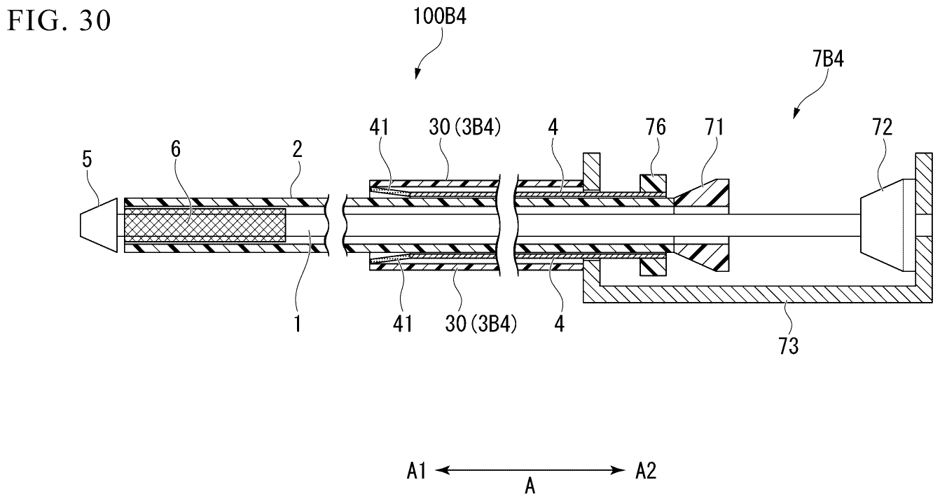

FIGS. 30 to 33 are diagrams representing a stent delivery device 100B4 that is another form of the present embodiment. The stent delivery device 100B4 includes the inner sheath 1, the outer sheath 2, an outermost layer sheath 3B4, a self-expanding sheath 4, the tip 5, the stent 6, and an operation portion 7B4.

The outermost layer sheath 3B4 has the outermost layer sheath main body 30 but does not have the cutout portion 31 and the stretchable portion 32.

The self-expanding sheath 4 is disposed between the outer sheath 2 and the outermost layer sheath 3B4. The self-expanding sheath 4 can advance and retract in the longitudinal direction A. If the self-expanding sheath 4 advances, a self-expanding portion 41 provided at a leading end of the self-expanding sheath 4 protrudes from the outermost layer sheath 3B4. The self-expanding portion 41 protruding from the outermost layer sheath 3B4 expands outward in the radial direction R due to a self-expanding force.

The operation portion 7B2 has the outer operation handle 71, the inner operation handle 72, the casing 73, and a self-expanding sheath operation handle 76. The proximal end of the self-expanding sheath 4 is attached to the self-expanding sheath operation handle 76.

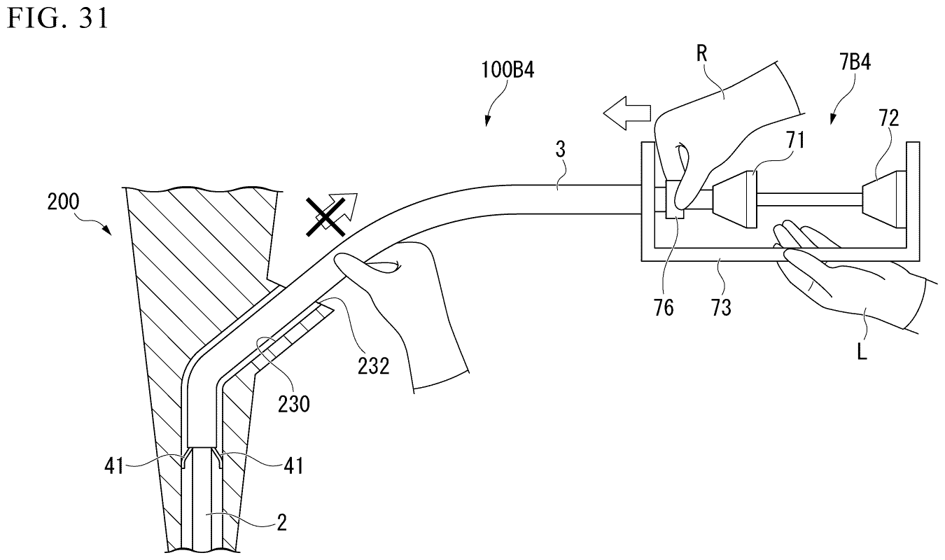

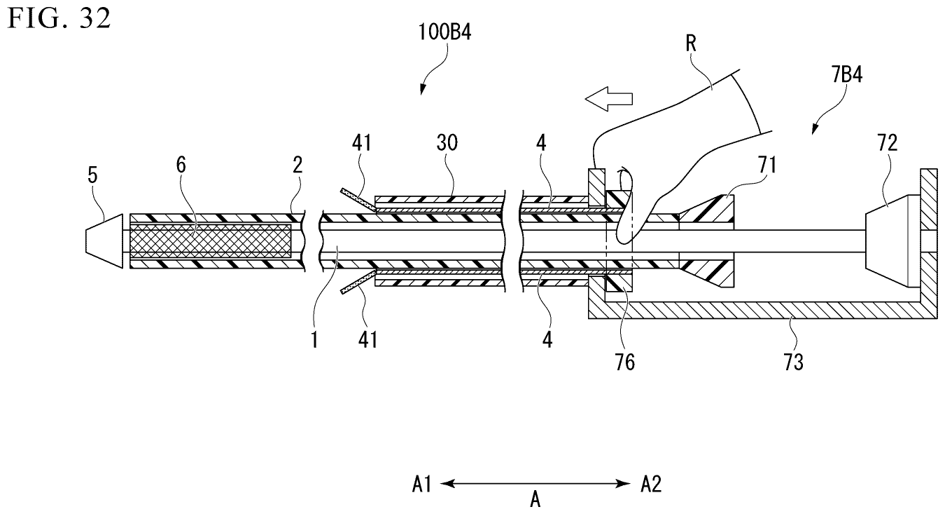

As represented in FIGS. 31 and 32, if the helper advances the self-expanding sheath operation handle 76 with the right hand R, the self-expanding sheath 4 advances, and the self-expanding portion 41 protrudes from the outermost layer sheath 3B4. The self-expanding portion 41 presses the treatment tool channel 230 from the inward side by expanding outward in the radial direction R. As a result, the outermost layer sheath 3B4 is fixed to the treatment tool channel 230, and the inner sheath 1 is fixed to the treatment tool channel 230.

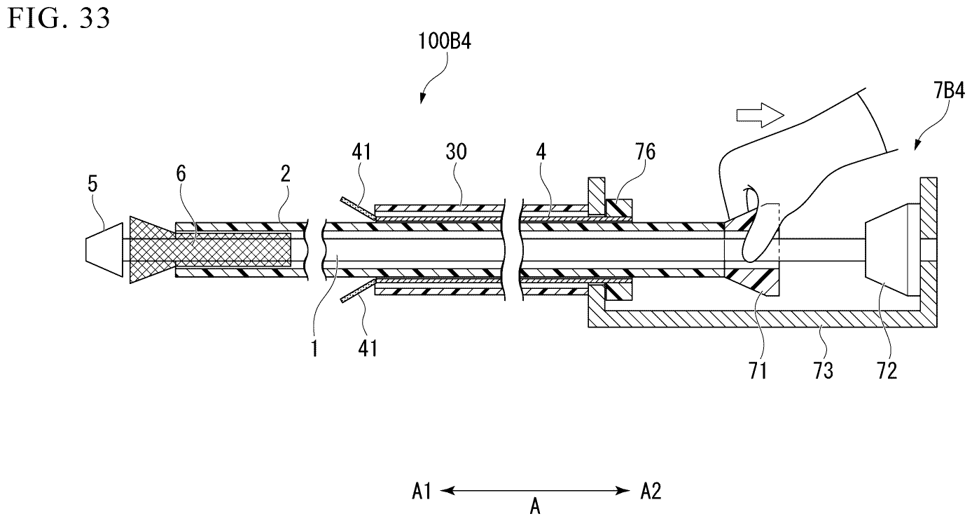

As represented in FIG. 33, the helper indwells the stent 6 by retracting the outer operation handle 71 with the right hand R.

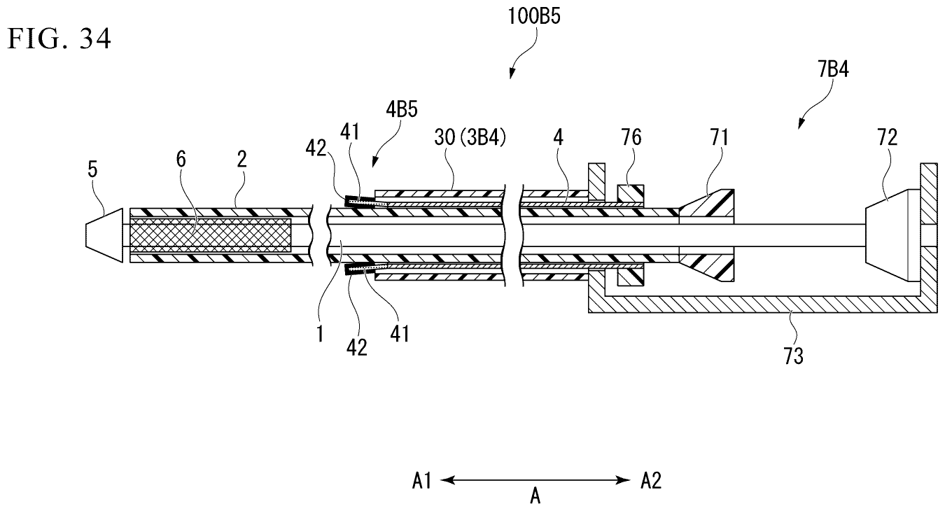

FIGS. 34 to 36 are diagrams representing a stent delivery device 100B5 that is another form of the present embodiment. The stent delivery device 100B5 includes the inner sheath 1, the outer sheath 2, the outermost layer sheath 3B4, a self-expanding sheath 4B5, the tip 5, the stent 6, and the operation portion 7B4.

Similar to the self-expanding sheath 4, the self-expanding sheath 4B5 has the self-expanding portion 41. The self-expanding sheath 4B5 further has a marker 42 attached to the self-expanding portion 41. The marker 42 is formed to be thicker than the self-expanding portion 41. For this reason, when the operator attempts to pull the outer sheath 2 while the self-expanding portion 41 is in a state of not self-expanding, the outer sheath 2 is not pulled due to a frictional force generated between the marker 42 and the outer sheath 2. The self-expanding sheath 4B5 can curb a situation in which the outer sheath 2 is pulled and the stent 6 is released before the inner sheath 1 is fixed to the treatment tool channel 230 by the self-expanding portion 41.

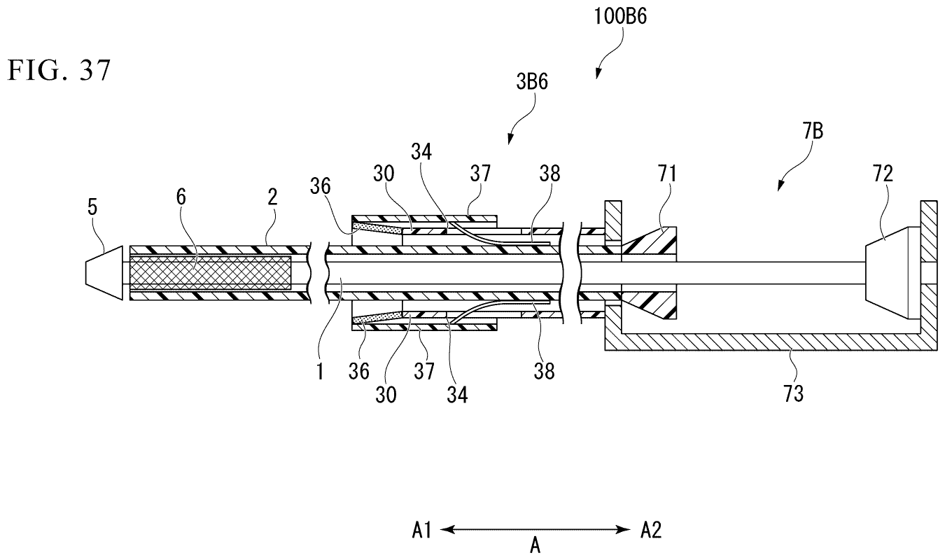

FIGS. 37 to 39 are diagrams representing a stent delivery device 100B6 that is another form of the present embodiment. The stent delivery device 100B6 includes the inner sheath 1, the outer sheath 2, an outermost layer sheath 3B6, the tip 5, the stent 6, and the operation portion 7B.

The outermost layer sheath 3B6 has the outermost layer sheath main body 30 provided with the slit 34 lying in the longitudinal direction A, a self-expanding portion 36 provided at a leading end of the outermost layer sheath main body 30, a cover member 37 attached to be able to advance and retract on the outward side of the outermost layer sheath main body 30, and the wire 35. The wire 35 is inserted through the slit 34, the leading end is attached to the cover member 37, and the proximal end is attached to the outer circumferential surface of the outer sheath 2.

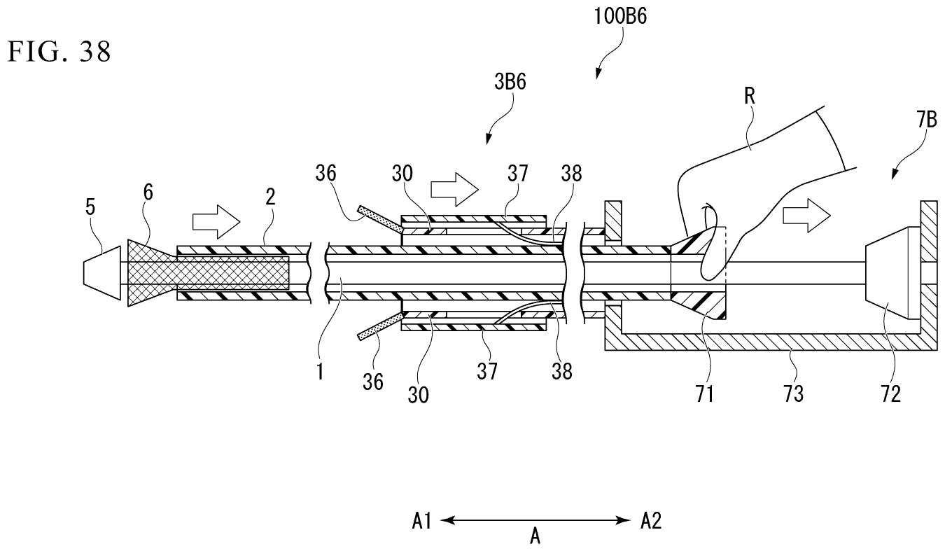

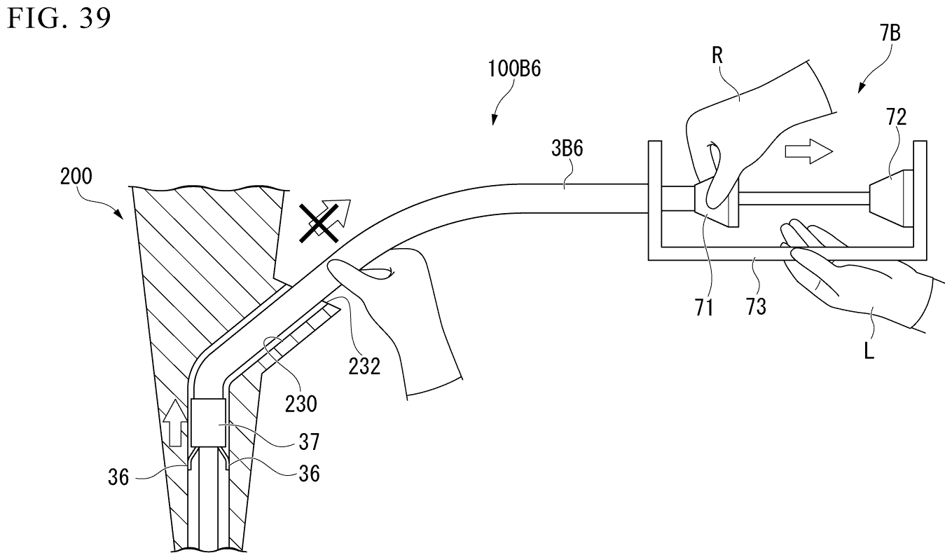

As represented in FIGS. 38 and 39, if the helper retracts the outer operation handle 71 with the right hand R, the outer sheath 2 retracts and the wire 35 is pulled. As a result, the cover member 37 retracts in conjunction therewith so that the self-expanding portion 36 protrudes from the outermost layer sheath 3B4. The self-expanding portion 36 presses the treatment tool channel 230 from the inward side by expanding outward in the radial direction R. As a result, the outermost layer sheath 3B6 is fixed to the treatment tool channel 230, and the inner sheath 1 is fixed to the treatment tool channel 230.

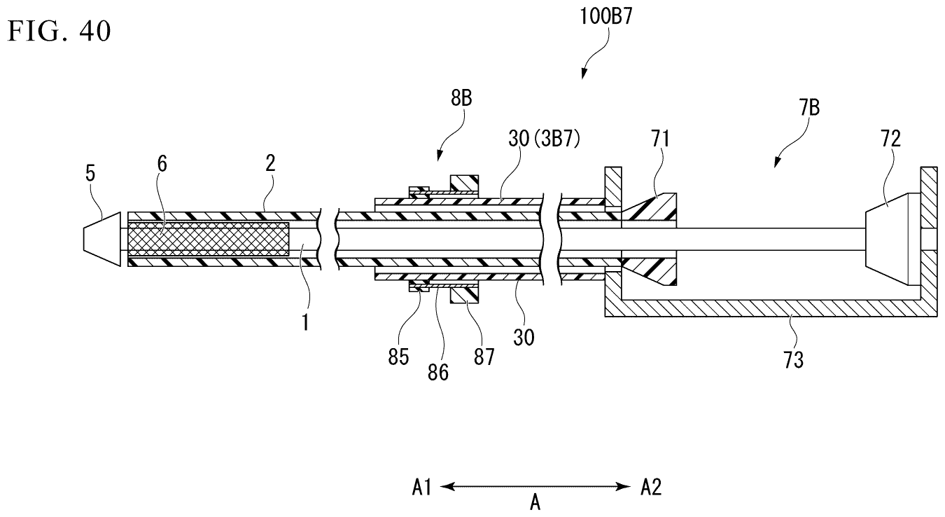

FIGS. 40 to 42 are diagrams representing a stent delivery device 100B7 that is another form of the present embodiment. The stent delivery device 100B7 includes the inner sheath 1, the outer sheath 2, an outermost layer sheath 3B7, the tip 5, the stent 6, the operation portion 7B, and a rubber plug 8B.

The outermost layer sheath 3B7 has the outermost layer sheath main body 30 but does not have the cutout portion 31 and the stretchable portion 32.

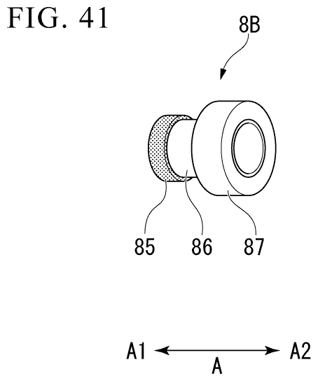

As represented in FIG. 41, the rubber plug (engagement member) 8B has a rubber material 85, a cylindrical main body 86, and a holder 87. The rubber material 85 is provided at a leading end of the main body 86 and can be attached and detached with respect to the forceps opening 232. The main body 86 is attached to an outer circumferential portion of the outermost layer sheath main body 30. The holder 87 is provided at a proximal end of the main body 86.

As represented in FIG. 42, when the stent delivery device 100B7 is inserted into the treatment tool channel 230, the rubber plug 8B is attached to the forceps opening 232. As a result, the outermost layer sheath main body 30 is fixed to the treatment tool channel 230, and the inner sheath 1 is fixed to the treatment tool channel 230.

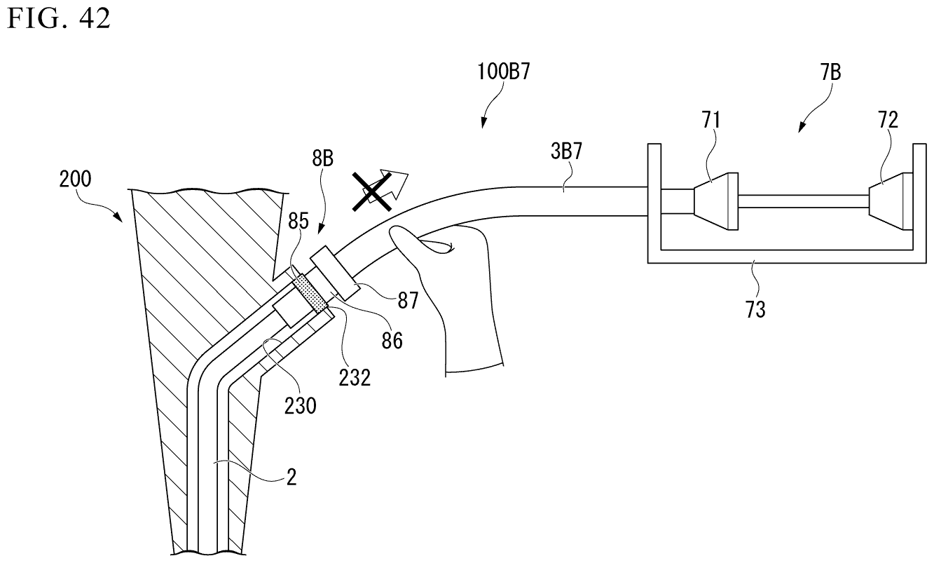

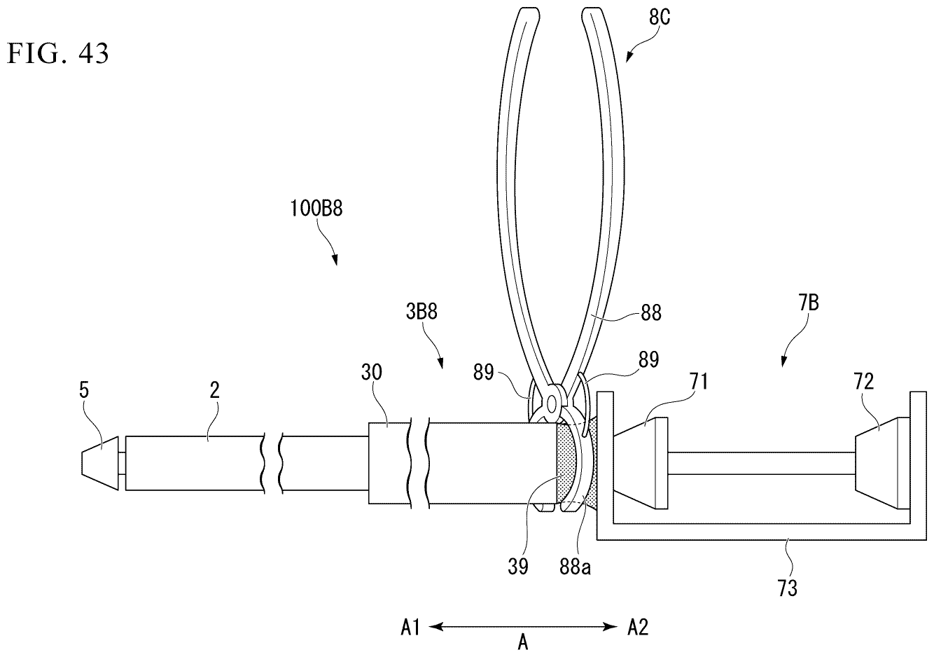

FIGS. 43 to 48 are diagrams representing a stent delivery device 100B8 that is another form of the present embodiment. The stent delivery device 100B8 includes the inner sheath 1, the outer sheath 2, an outermost layer sheath 3B8, the tip 5, the stent 6, the operation portion 7B, and a pinching member 8C.

The outermost layer sheath 3B8 has the outermost layer sheath main body 30, and a tubular rubber member 39 coupling the outermost layer sheath main body 30 and the casing 73.

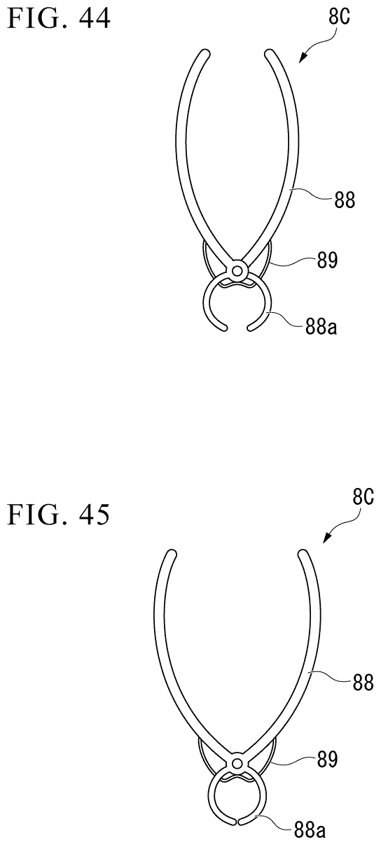

As represented in FIGS. 44 and 45, the pinching member (engagement member) 8C has a plier-shaped main body 88 and an elastic member 89 biasing clamping portions 88a of the main body 88 in a closed state. As represented in FIG. 43, the clamping portions 88a are disposed so as to sandwich the rubber member 39 therebetween.

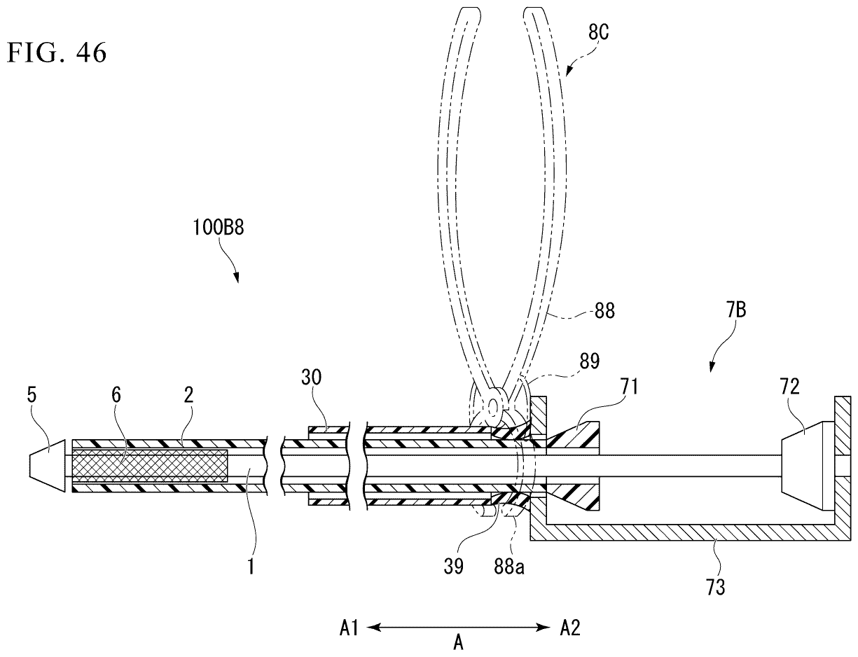

As represented in FIG. 46, the clamping portions 88a sandwiching the rubber member 39 therebetween increase a frictional force generated between the rubber member 39 and the outer sheath 2. The clamping portions 88a sandwiching the rubber member 39 therebetween hinder retracting movement of the outer sheath 2 and function as stoppers with respect to an operation of releasing the stent 6.

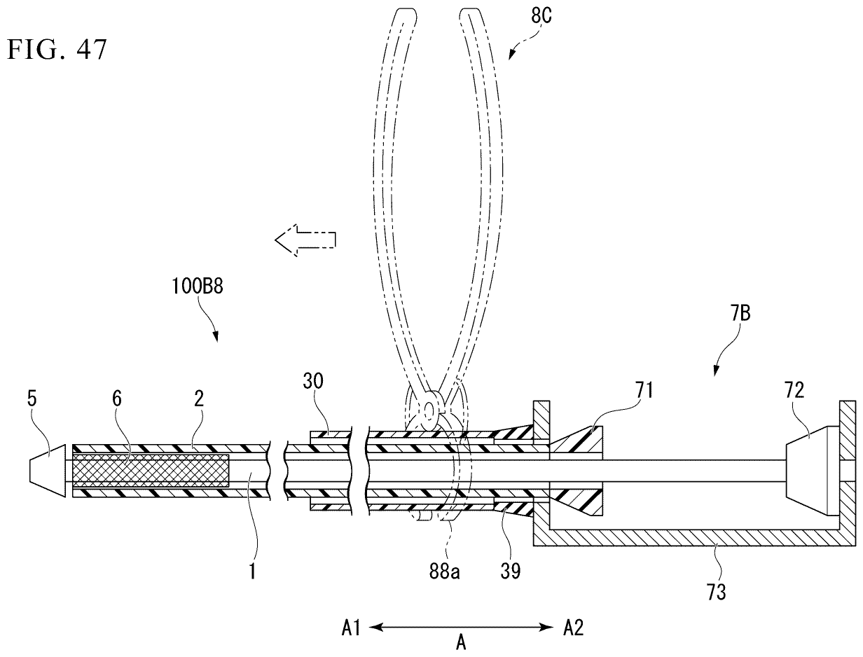

As represented in FIGS. 47 and 48, the user removes the clamping portions 88a of the pinching member 8C from the rubber member 39 and advances them along the outer sheath 2. The user attaches the clamping portions 88a of the pinching member 8C to the forceps opening 232 and fixes the endoscope 200 and the outer sheath 2. As a result, the outermost layer sheath main body 30 is fixed to the treatment tool channel 230, and the inner sheath 1 is fixed to the treatment tool channel 230.

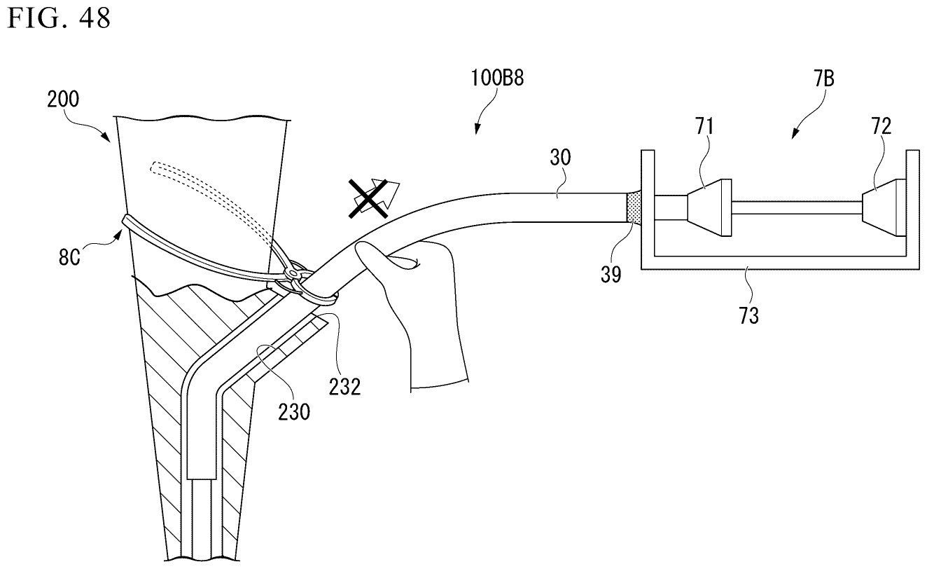

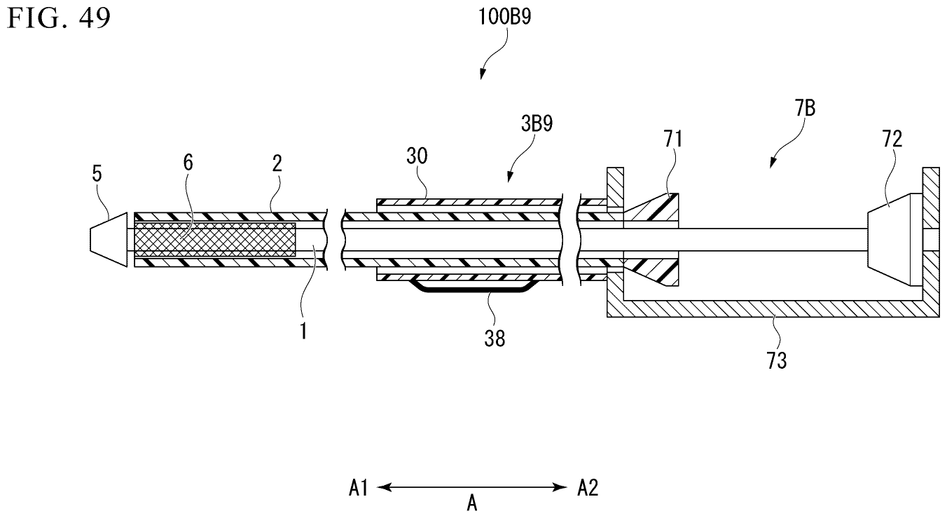

FIGS. 49 and 50 are diagrams representing a stent delivery device 100B9 that is another form of the present embodiment. The stent delivery device 100B9 includes the inner sheath 1, the outer sheath 2, an outermost layer sheath 3B9, the tip 5, the stent 6, the operation portion 7B, and a locking device 8D.

The outermost layer sheath 3B9 has the outermost layer sheath main body 30, and a wire 38 attached to the outermost layer sheath main body 30.

For example, the locking device (engagement member) 8D is a guide wire locking device for fixing the guide wire GW to the endoscope 200.

As represented in FIG. 50, the user attaches the locking device 8D to a part in the vicinity of the forceps opening 232 of the endoscope 200 and fixes the wire 38 using the locking device 8D. As a result, the outermost layer sheath main body 30 is fixed to the treatment tool channel 230, and the inner sheath 1 is fixed to the treatment tool channel 230.

According to the stent delivery device 100B and the like of the present embodiment, the location of the inner sheath 1 with respect to the treatment tool channel 230 is fixed, and therefore the location where the stent 6 is stored is unlikely to deviate from a target location.

Hereinabove, the second embodiment of the present invention has been described in detail with reference to the drawings, but the specific constitutions are not limited to this embodiment, and design change and the like within a range not departing from the gist of the present invention are also included. In addition, the constituent elements described in the foregoing embodiment and the following modification examples can be suitably constituted in a combination.

### Third Embodiment

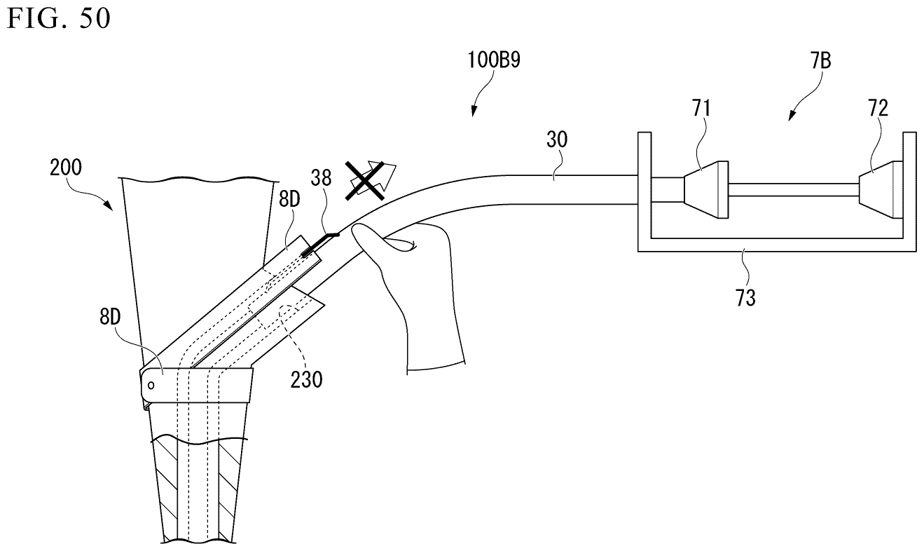

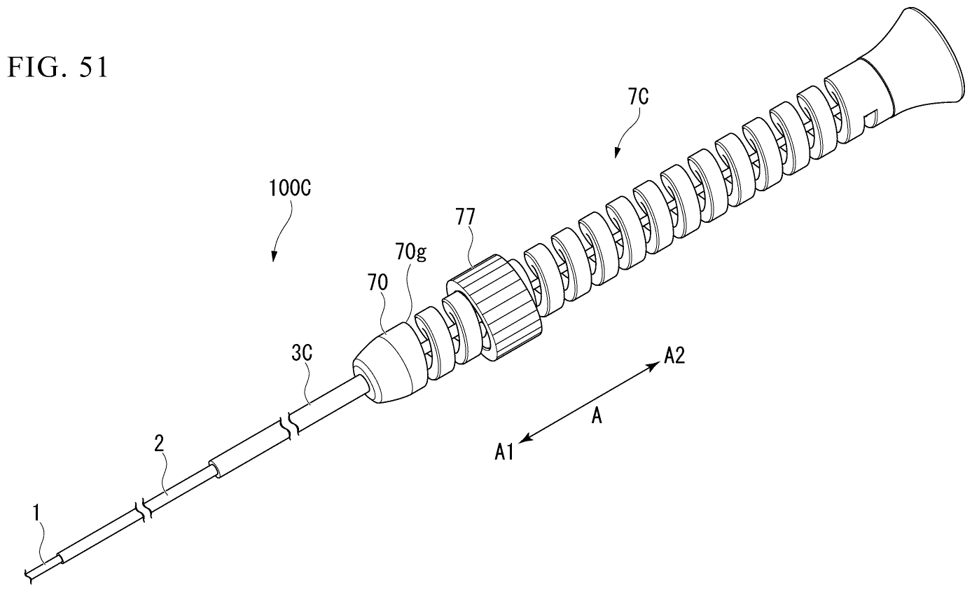

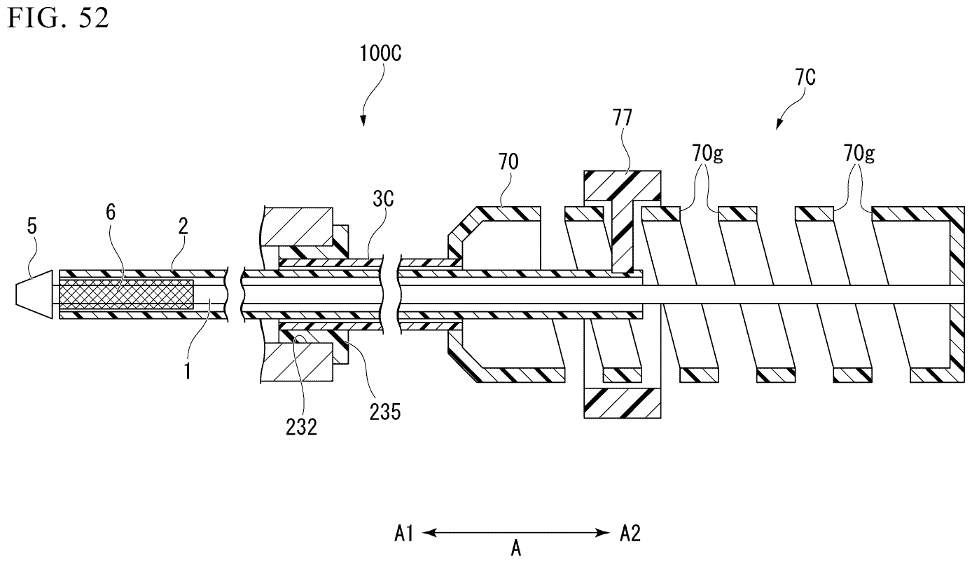

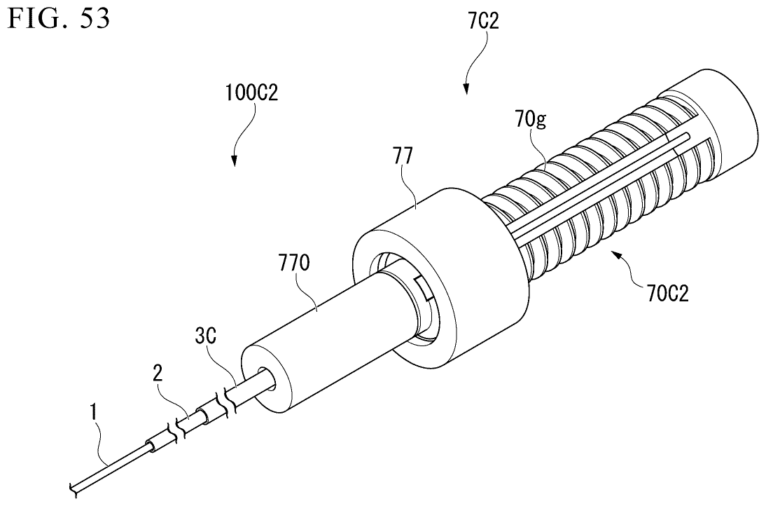

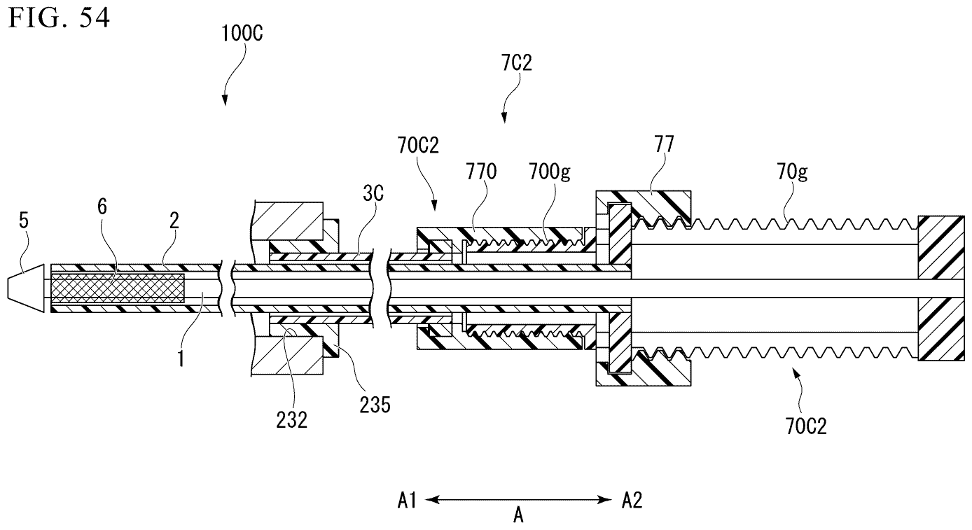

A third embodiment of the present invention will be described with reference to FIGS. 51 to 58. In the following description, the same reference signs are applied to common constituents which have already been described, and duplicate description will be omitted.

FIG. 51 is a diagram representing a stent delivery device 100C according to the present embodiment.

Similar to the stent delivery device 100 of the first embodiment, the stent delivery device 100C is inserted through the treatment tool channel 230 of the endoscope 200.

The stent delivery device 100C is formed to be thin and long in its entirety and includes the inner sheath (first sheath) 1, the outer sheath (second sheath) 2, an outermost layer sheath (third sheath) 3C, the tip 5, the stent 6, and an operation portion 7C. The stent delivery device 100C has a triple sheath structure in which the inner sheath 1, the outer sheath 2, and the outermost layer sheath 3C overlap.

FIG. 52 is a cross-sectional diagram of the stent delivery device 100C.

The outermost layer sheath 3C has the outermost layer sheath main body 30. A proximal end of the outermost layer sheath main body 30 is attached to the operation portion 7C. When the stent delivery device 100C is used by being inserted into the treatment tool channel 230 of the endoscope 200, similar to the outermost layer sheath 3 of the second embodiment, a leading end of the outermost layer sheath 3C is disposed in a part which is inserted into the treatment tool channel 230.

The operation portion 7C has an operation portion main body 70 and a dial 77. The inner sheath 1 is attached to a proximal end portion of the operation portion main body 70. The operation portion main body 70 is formed to have a substantially tubular shape and has a spiral groove 70g formed to have a spiral shape in the outer circumferential portion.

The dial 77 is a tubular dial attached to an outer circumferential portion of the operation portion main body 70. The dial 77 can advance and retract in the longitudinal direction A while rotating along the spiral groove 70g of the operation portion main body 70. The dial 77 is attached to the outer sheath 2 inside the operation portion main body 70. The user can retract the outer sheath 2 and release the stent 6 by rotating the dial 77 with respect to the operation portion main body 70.

A force applied to the operation portion 7C by the user in order to retract the outer sheath 2 and indwell the stent 6 becomes a rotational moment in the circumferential direction C with respect to the longitudinal direction A. For this reason, a force for advancing and retracting the stent delivery device 100C is not applied, the location of the inner sheath 1 is unlikely to deviate, and the location where the stent 6 is stored is unlikely to deviate from a target location.

Since a force applied to the operation portion 7C by the user is a rotational moment as described above, a force required to rotate the dial 77 can be reduced by increasing the size of the dial 77.

The dial 77 moves while rotating along the spiral groove 70g. For this reason, the user can accurately control release of the stent 6 by finely adjusting advancing/retracting movement of the outer sheath 2. For example, the spiral groove 70g is set such that the outer sheath moves 2 mm for each rotation of the dial 77.

FIGS. 53 to 56 are diagrams representing a stent delivery device 100C2 that is another form of the present embodiment. The stent delivery device 100C2 includes the inner sheath 1, the outer sheath 2, the outermost layer sheath 3C, the tip 5, the stent 6, and an operation portion 7C2.

The operation portion 7C2 has an operation portion main body 70C2, the dial 77, and an auxiliary dial 770. The operation portion main body 70C2 is formed to have substantially a tubular shape and has the spiral groove 70g and an auxiliary spiral groove 700g formed to have a spiral shape in the outer circumferential portion. The auxiliary spiral groove 700g is a groove with which the auxiliary dial 770 engages.

The dial 77 and the auxiliary dial 770 are tubular dials attached to the operation portion main body 70C2. The dial 77 is attached to the outer sheath 2. The auxiliary dial 770 is attached to the outermost layer sheath 3C. The dial 77 is attached to the proximal end side from the auxiliary dial 770. The outer diameter of the dial 77 is larger than the outer diameter of the auxiliary dial 770.

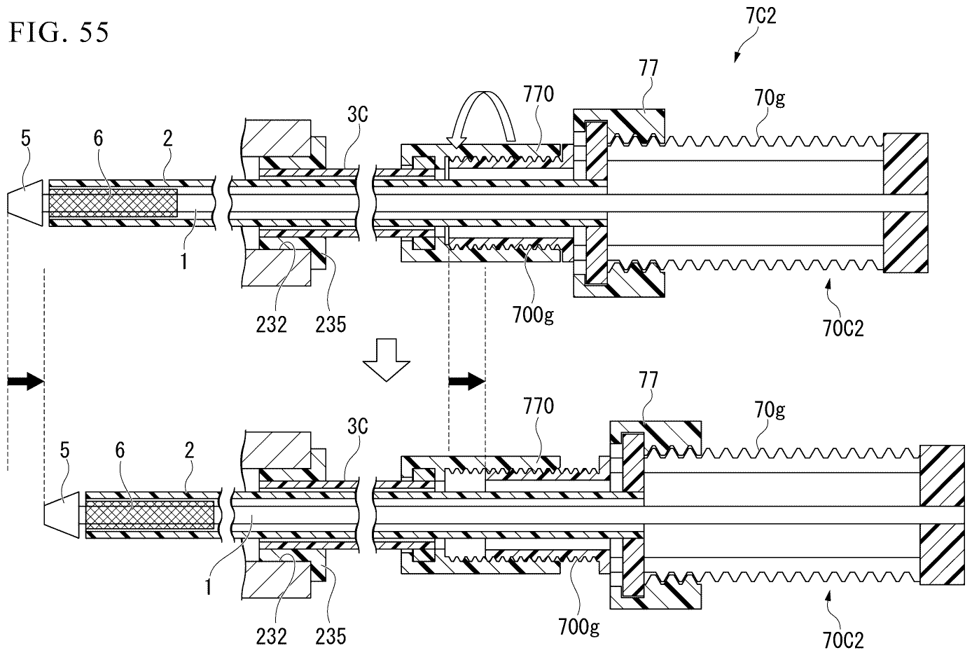

As represented in FIG. 55, the inner sheath 1 and the outer sheath 2 can be advanced and retracted with respect to the outermost layer sheath 3C by rotating the auxiliary dial 770 with respect to the operation portion main body 70C2. The user can adjust a leading end location of the stent delivery device 100C2 by rotating the auxiliary dial 770.

As represented in FIG. 56, the outer sheath 2 can be advanced and retracted with respect to the inner sheath 1 by rotating the dial 77 with respect to the operation portion main body 70C2. The user can retract the outer sheath 2 and release the stent 6 by rotating the dial 77.

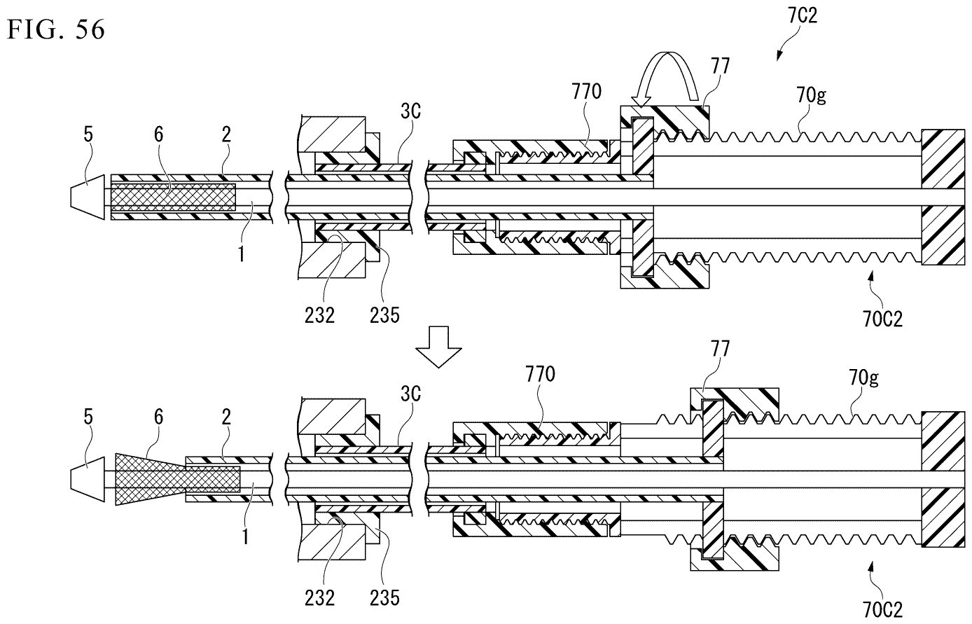

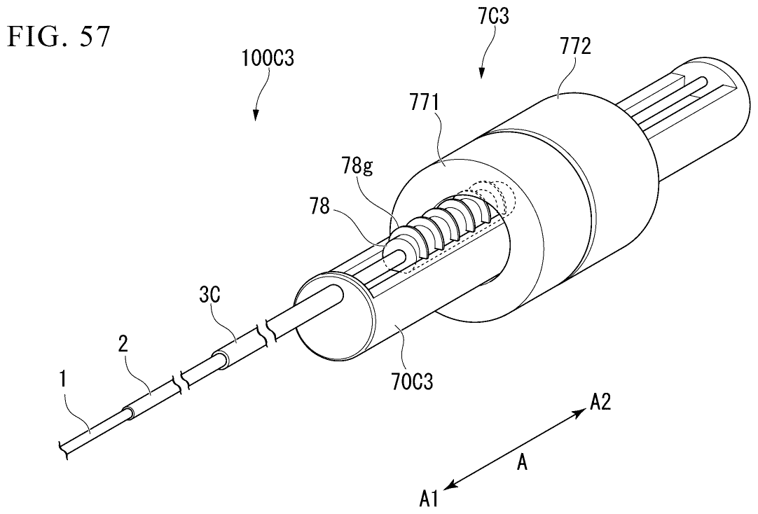

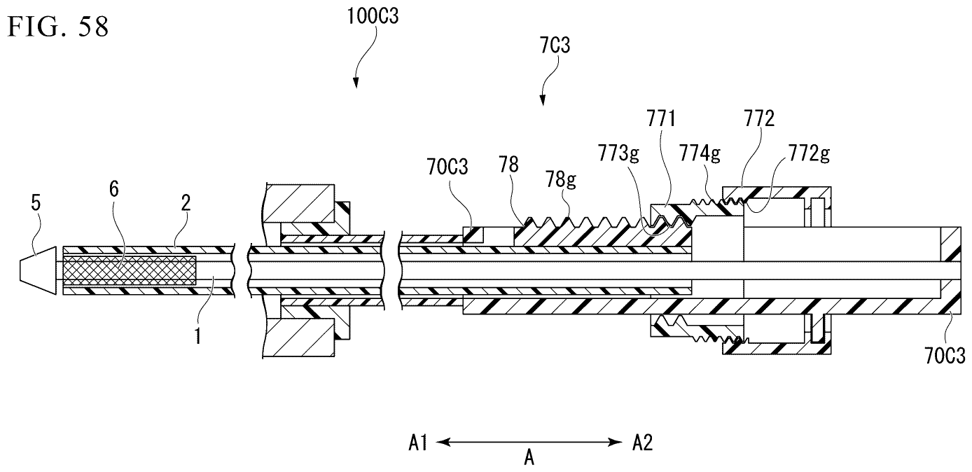

FIGS. 57 to 58 are diagrams representing a stent delivery device 100C3 that is another form of the present embodiment. The stent delivery device 100C3 includes the inner sheath 1, the outer sheath 2, the outermost layer sheath 3C, the tip 5, the stent 6, and an operation portion 7C3.

The operation portion 7C3 has an operation portion main body 70C3, a first dial 771, a second dial 772, and an outer movable portion 78. The operation portion main body 70C3 is formed to have a substantially tubular shape.

The outer movable portion 78 has a second spiral groove 78g which is attached to the outer circumferential surface of the outer sheath 2 and is formed to have a spiral shape.

The first dial 771 is a tubular dial attached to the operation portion main body 70C3. A third spiral groove 773g which engages with the second spiral groove 78g of the outer movable portion 78 is formed on the inward side of the first dial 771 in the radial direction R. A fourth spiral groove 774g which engages with a fifth spiral groove 772g of the second dial 772 is formed on the outward side of the first dial 771 in the radial direction R.

The second dial 772 is a tubular dial attached to the operation portion main body 70C3. A proximal end of the second dial 772 is fixed to the operation portion main body 70C3. The fifth spiral groove 772g which engages with the fourth spiral groove 774g of the first dial 771 is formed on the inward side of the second dial 772 in the radial direction R.

The pitches of the second spiral groove 78g and the third spiral groove 773g are larger than the pitches of the fourth spiral groove 774g and the fifth spiral groove 772g. For this reason, the outer sheath 2 more significantly advances and retracts when the first dial 771 is rotated than when the second dial 772 is rotated. On the contrary, the outer sheath 2 can be moved more slowly when the second dial 772 is rotated than when the first dial 771 is rotated.

According to the stent delivery device 100C and the like of the present embodiment, the location of the inner sheath 1 is unlikely to deviate, and the location where the stent 6 is stored is unlikely to deviate from a target location.

Hereinabove, the third embodiment of the present invention has been described in detail with reference to the drawings, but the specific constitutions are not limited to this embodiment, and design change and the like within a range not departing from the gist of the present invention are also included. In addition, the constituent elements described in the foregoing embodiment and the following modification examples can be suitably constituted in a combination.

### Fourth Embodiment

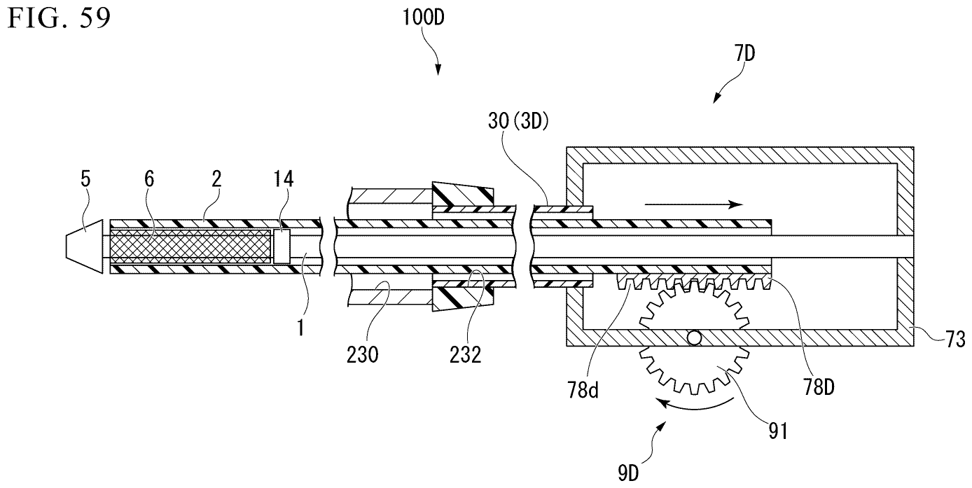

A fourth embodiment of the present invention will be described with reference to FIGS. 59 to 67. In the following description, the same reference signs are applied to common constituents which have already been described, and duplicate description will be omitted.

FIG. 59 is a diagram representing a stent delivery device 100D according to the present embodiment.

Similar to the stent delivery device 100 of the first embodiment, the stent delivery device 100D is inserted through the treatment tool channel 230 of the endoscope 200.

The stent delivery device 100D is formed to be thin and long in its entirety and includes the inner sheath (first sheath) 1, the outer sheath (second sheath) 2, an outermost layer sheath (third sheath) 3D, the tip 5, the stent 6, and an operation portion 7D. The stent delivery device 100D has a triple sheath structure in which the inner sheath 1, the outer sheath 2, and the outermost layer sheath 3D overlap.

The outermost layer sheath 3D has the outermost layer sheath main body 30. The proximal end of the outermost layer sheath main body 30 is attached to the operation portion 7D. When the stent delivery device 100D is used by being inserted into the treatment tool channel 230 of the endoscope 200, similar to the outermost layer sheath 3 of the second embodiment, a leading end of the outermost layer sheath 3D is disposed in a part which is inserted into the treatment tool channel 230.

The operation portion 7D has the casing 73, an outer movable portion 78D, and a drive portion 9D driving the outer sheath 2. The operation portion 7D does not have the outer operation handle 71 and the inner operation handle 72.

The outer movable portion 78D is attached to the outer sheath 2 inside the casing 73. The outer movable portion 78D has a driven gear 78d.

The drive portion 9D has a first driving gear 91. The first driving gear 91 is attached to the casing 73, engages with the driven gear 78d, and drives the driven gear 78d. The user can advance and retract the driven gear 78d in the longitudinal direction A and can advance and retract the outer sheath 2 by rotating the first driving gear 91 with respect to the casing 73.

A force applied to the operation portion 7D by the user in order to retract the outer sheath 2 and indwell the stent 6 becomes a rotational moment with respect to the first driving gear 91. For this reason, a force for advancing and retracting the stent delivery device 100D is not applied, the location of the inner sheath 1 is unlikely to deviate, and the location where the stent 6 is stored is unlikely to deviate from a target location.

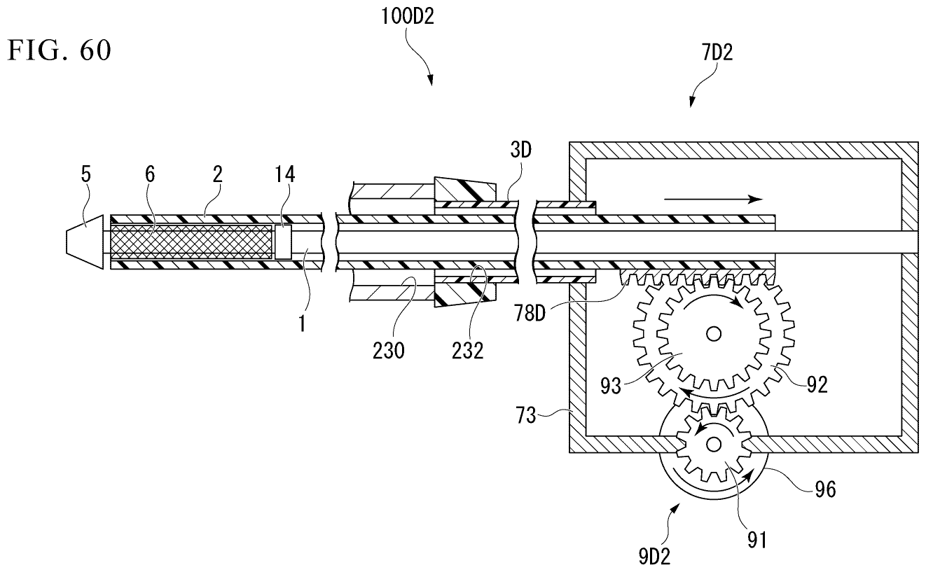

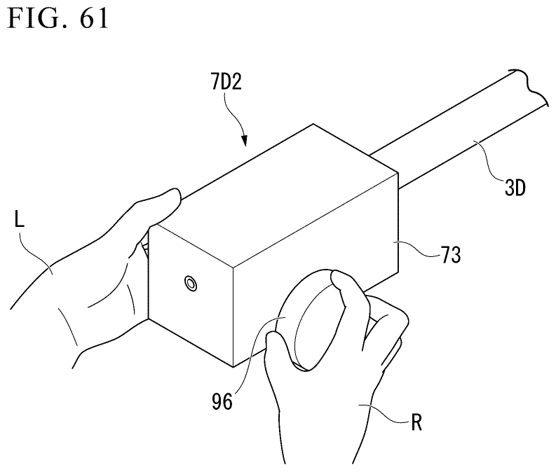

FIGS. 60 and 61 are diagrams representing a stent delivery device 100D2 that is another form of the present embodiment. The stent delivery device 100D2 includes the inner sheath 1, the outer sheath 2, the outermost layer sheath 3D, the tip 5, the stent 6, and an operation portion 7D2.

The operation portion 7D2 has the casing 73, the outer movable portion 78D, and a drive portion 9D2 driving the outer sheath 2.

The drive portion 9D2 is constituted of a multi-stage reduction gear, which has the first driving gear 91, a second driving gear 92, and a third driving gear 93. The constitution of the multi-stage reduction gear of the drive portion 9D2 is not limited to this.

The second driving gear 92 and the third driving gear 93 are multi-stage gears which integrally rotate. The second driving gear 92 is larger than the third driving gear 93 and is driven by the first driving gear 91. The third driving gear 93 drives the outer movable portion 78D.

A driving handle 96 is attached to the first driving gear 91. The driving handle 96 has a larger outer diameter than the first driving gear 91. For this reason, the driving handle 96 and the first driving gear 91 constitute a speed reducer.

The drive portion 9D2 has a multi-stage reduction gear constitution so that the user can advance and retract the outer sheath 2 with a smaller force.

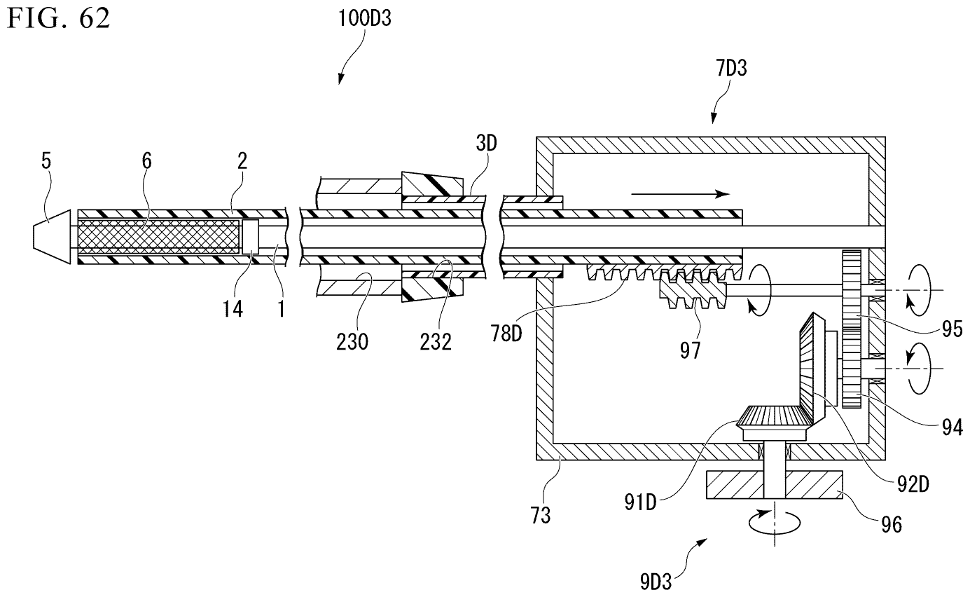

FIG. 62 is a diagram representing a stent delivery device 100D3 that is another form of the present embodiment. The stent delivery device 100D3 includes the inner sheath 1, the outer sheath 2, the outermost layer sheath 3D, the tip 5, the stent 6, and an operation portion 7D3.

The operation portion 7D3 has the casing 73, the outer movable portion 78D, and a drive portion 9D3 driving the outer sheath 2.

The drive portion 9D3 is constituted of a multi-stage reduction gear, which has a first driving gear 91D, a second driving gear 92D, a fourth driving gear 94, a fifth driving gear 95, the driving handle 96, and a worm gear 97. The first driving gear 91D and the second driving gear 92D are bevel gears. The first driving gear 91D drives the second driving gear 92D. The fourth driving gear 94 individually rotates together with the second driving gear 92D and drives the fifth driving gear 95. The worm gear 97 individually rotates together with the fifth driving gear 95 and drives the outer movable portion 78D. Since the drive portion 9D3 uses the worm gear 97, even if the operator stops his/her hands in the middle of releasing, the driving handle 96 does not reversely rotate upon reception of a force of the elastically deformed outer sheath 2 which tends to return to its original state. For this reason, the operator can instantly perform an accurate releasing operation when restarting an operation. Since the drive portion 9D3 uses the bevel gears and the worm gear 97, it has a high degree of freedom in terms of gear constitution. If the worm gear 97 can be disposed at an appropriate location, the bevel gears are not essential.

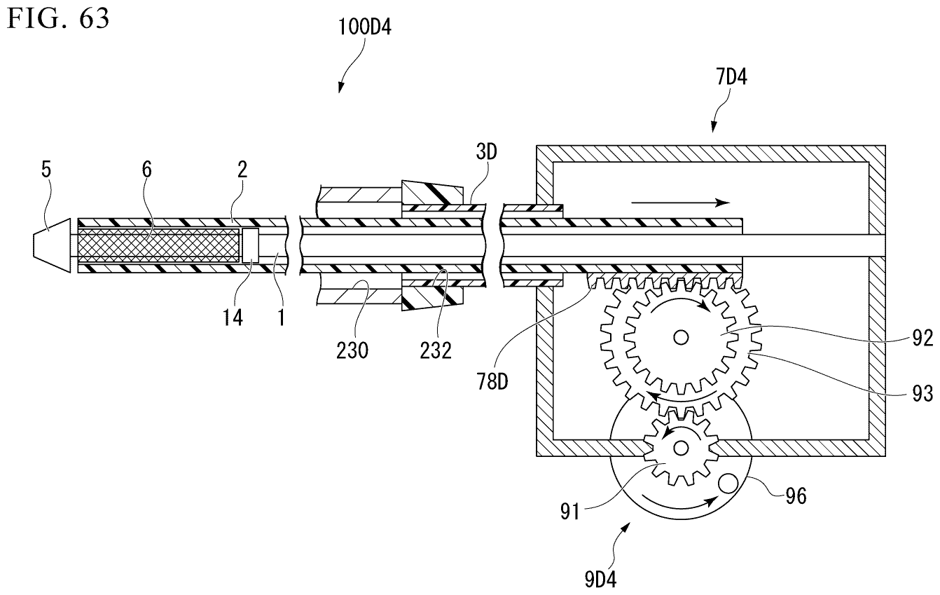

FIGS. 63 to 65 are diagrams representing a stent delivery device 100D4 that is another form of the present embodiment. The stent delivery device 100D4 includes the inner sheath 1, the outer sheath 2, the outermost layer sheath 3D, the tip 5, the stent 6, and an operation portion 7D4.

The operation portion 7D4 has the casing 73, the outer movable portion 78D, and a drive portion 9D4 driving the outer sheath 2.

The drive portion 9D4 is constituted of a multi-stage reduction gear, which has the first driving gear 91, the second driving gear 92, the third driving gear 93, the driving handle (fine adjustment handle) 96, and a coarse adjustment handle 96h. The drive portion 9D2 has two handles (the fine adjustment handle 96 and the coarse adjustment handle 96h) with different torque transmission efficiency. The user can quickly and roughly operate the outer sheath 2 using the coarse adjustment handle 96h and can accurately operate the outer sheath 2 using the driving handle (fine adjustment handle) 96.

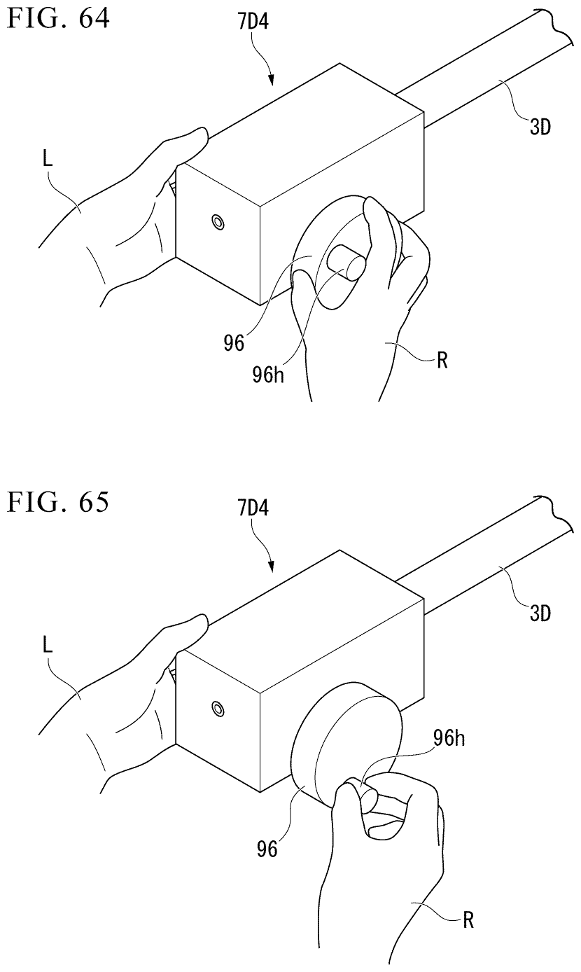

Since the driving handle (fine adjustment handle) 96 is grasped with all the fingers of the hand (FIG. 64), the torque is dispersed by the number of fingers. Although the coarse adjustment handle 96h can be grasped with only some of the fingers, the torque is likely to concentrate at one point with respect to the coarse adjustment handle 96h (FIG. 65). It is easy for the operator to switch between accurate release of the stent 6 and quick release of the stent 6 by simply switching the operating handle from one to the other.

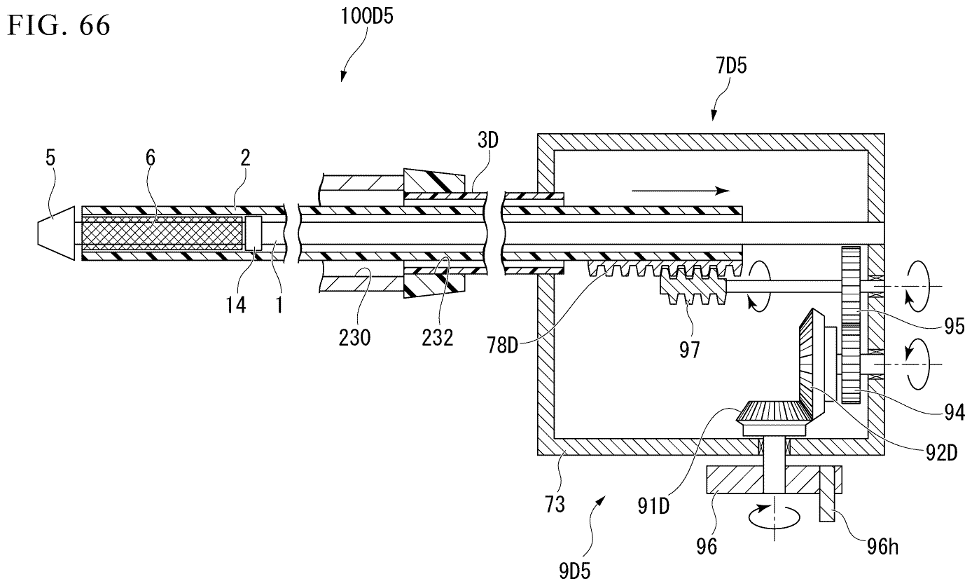

FIG. 66 is a diagram representing a stent delivery device 100D5 that is another form of the present embodiment. The stent delivery device 100D5 includes the inner sheath 1, the outer sheath 2, the outermost layer sheath 3D, the tip 5, the stent 6, and an operation portion 7D5.

The operation portion 7D5 has the casing 73, the outer movable portion 78D, and a drive portion 9D5 driving the outer sheath 2.

The drive portion 9D5 further has the coarse adjustment handle 96h in addition to the drive portion 9D3. The user can quickly and roughly operate the outer sheath 2 using the coarse adjustment handle 96h and can accurately operate the outer sheath 2 using the driving handle (fine adjustment handle) 96. It is easy for the operator to switch between accurate release of the stent 6 and quick release of the stent 6 by simply switching the operating handle from one to the other. In addition, since the drive portion 9D5 uses the worm gear 97, even if the operator stops his/her hands in the middle of releasing, the driving handle 96 does not reversely rotate upon reception of a force of the elastically deformed outer sheath 2 which tends to return to its original state. For this reason, the operator can instantly perform an accurate releasing operation when restarting an operation.

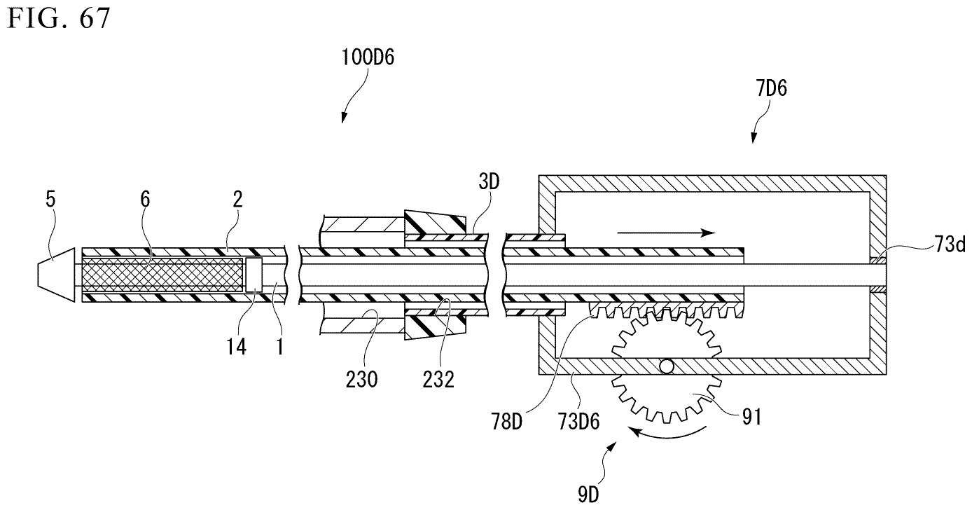

FIG. 67 is a diagram representing a stent delivery device 100D6 that is another form of the present embodiment. The stent delivery device 100D6 includes the inner sheath 1, the outer sheath 2, the outermost layer sheath 3D, the tip 5, the stent 6, and an operation portion 7D6.

The operation portion 7D6 has a casing 73D6, the outer movable portion 78D, and the drive portion 9D driving the outer sheath 2. The casing 73D6 has an inner lock 73d capable of fixing the inner sheath 1. The inner lock 73d is provided on a proximal end side of the casing 73D6. The user can clearly distinguish between the operation of advancing and retracting the inner sheath 1 and the outer sheath 2 (Operation 1) and the operation of releasing the stent 6 (Operation 2) by fixing and unfixing the inner sheath 1 using the inner lock 73d.

According to the stent delivery device 100D and the like of the present embodiment, the location of the inner sheath 1 is unlikely to deviate, and the location where the stent 6 is stored is unlikely to deviate from a target location. The inner lock 73d may be provided in the operation portion 7D2, the operation portion 7D3, the operation portion 7D4, and the operation portion 7D5 described above.

Hereinabove, the fourth embodiment of the present invention has been described in detail with reference to the drawings, but the specific constitutions are not limited to this embodiment, and design change and the like within a range not departing from the gist of the present invention are also included. In addition, the constituent elements described in the foregoing embodiment and the following modification examples can be suitably constituted in a combination.

### Fifth Embodiment

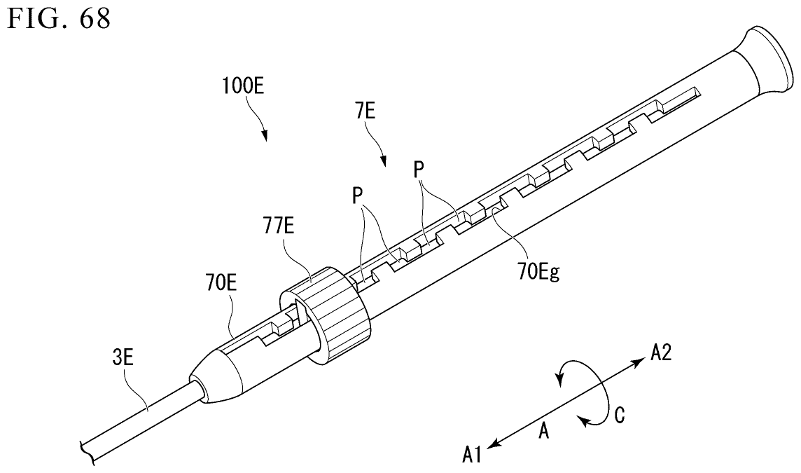

A fifth embodiment of the present invention will be described with reference to FIGS. 68 to 75. In the following description, the same reference signs are applied to common constituents which have already been described, and duplicate description will be omitted.

FIG. 68 is a diagram representing a stent delivery device 100E according to the present embodiment.

Similar to the stent delivery device 100 of the first embodiment, the stent delivery device 100E is inserted through the treatment tool channel 230 of the endoscope 200.

The stent delivery device 100E is formed to be thin and long in its entirety and includes the inner sheath (first sheath) 1, the outer sheath (second sheath) 2, an outermost layer sheath (third sheath) 3E, the tip 5, the stent 6, and an operation portion 7E. The stent delivery device 100E has a triple sheath structure in which the inner sheath 1, the outer sheath 2, and the outermost layer sheath 3E overlap.

The outermost layer sheath 3E has the outermost layer sheath main body 30. The proximal end of the outermost layer sheath main body 30 is attached to the operation portion 7E. When the stent delivery device 100E is used by being inserted into the treatment tool channel 230 of the endoscope 200, similar to the outermost layer sheath 3 of the second embodiment, a leading end of the outermost layer sheath 3E is disposed in a part which is inserted into the treatment tool channel 230.

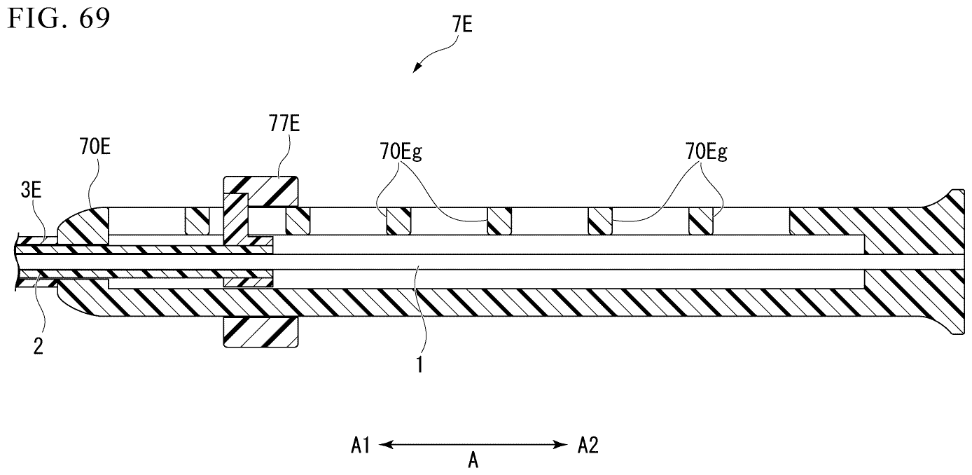

FIG. 69 is a cross-sectional diagram of the operation portion 7E.

The operation portion 7E has an operation portion main body 70E and a dial 77E. The inner sheath 1 is attached to a proximal end portion of the operation portion main body 70E. The operation portion main body 70E is formed to have substantially a tubular shape and has a bent groove 70Eg formed to have a zigzag shape in the outer circumferential portion. The bent groove 70Eg has bent spots P which bend with respect to the circumferential direction C with respect to the longitudinal direction A. In the present embodiment, the bent spots P are disposed at equal intervals in the longitudinal direction A. The bent groove 70Eg is formed to have a rectangular wave shape when viewed in the radial direction R.

The dial 77E is a tubular dial attached to an outer circumferential portion of the operation portion main body 70E. The dial 77E can advance and retract in the longitudinal direction A along the bent groove 70Eg of the operation portion main body 70E. The dial 77 is attached to the outer sheath 2 inside the operation portion main body 70. The user retracts the outer sheath 2 by advancing and retracting the dial 77 with respect to the operation portion main body 70 so that the stent 6 can be released.

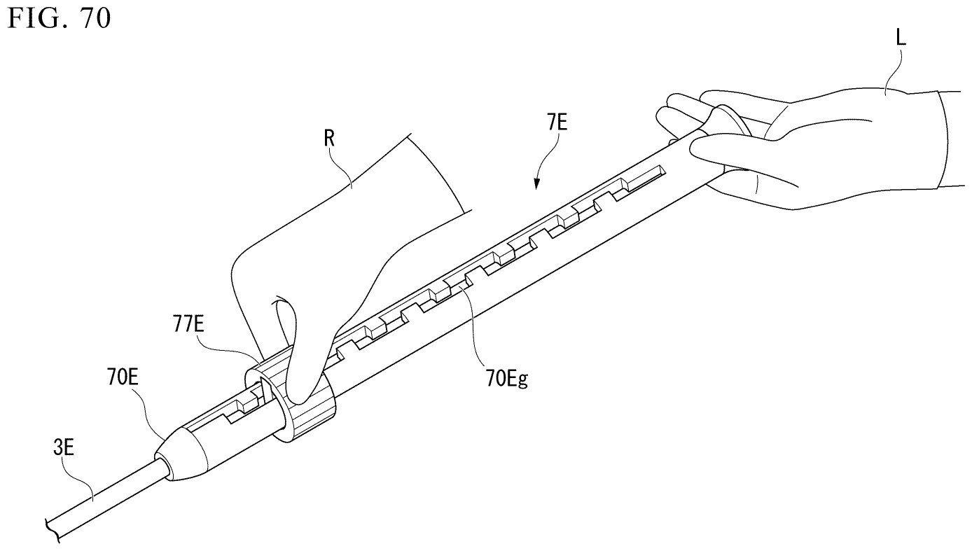

FIG. 70 is a diagram representing a method for using the operation portion 7E.

The user retracts the dial 77E and the outer sheath 2 in stages and releases the stent 6 by alternately performing a retracting movement operation of retracting the dial 77E to the bent spots P along the bent groove 70Eg and a rotation operation of moving the dial 77E in the circumferential direction C along the bent groove 70Eg. Since the outer sheath 2 can be retracted in stages, a situation in which the outer sheath 2 is retracted to an unintended location and the stent 6 is released can be curbed. The stent 6 can also be recaptured by advancing the dial 77E in stages.

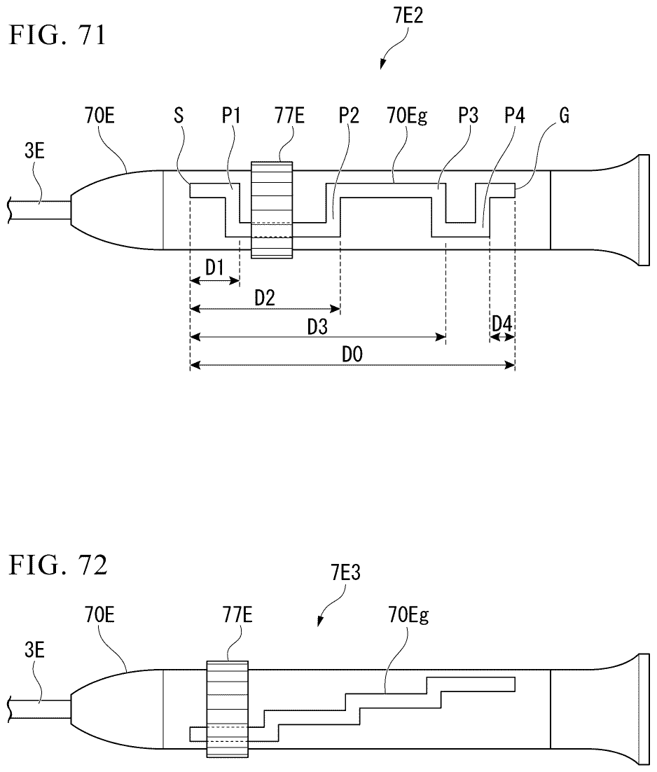

FIG. 71 is a diagram representing an operation portion 7E2 that is another form of the present embodiment.

The locations of the bent spots P in the bent groove 70Eg formed in the operation portion main body 70E of the operation portion 7E2 are associated with a step of releasing the stent 6.

The bent groove 70Eg extends from a leading end location S to a proximal end location G. When the dial 77E is disposed at the leading end location S of the bent groove 70Eg, the outer sheath 2 is not pulled at all. When the dial 77E is disposed at the proximal end location G of the bent groove 70Eg, the outer sheath 2 is pulled until the stent 6 is released. The bent spots P in the bent groove 70Eg will be referred to as a first bent spot P1, a second bent spot P2, a third bent spot P3, and a fourth bent spot P4 from the leading end side toward the proximal end side.

A distance D1 from the leading end location S to the first bent spot P1 in the longitudinal direction A is approximately 20% of a distance D from the leading end location S to the proximal end location G in the longitudinal direction A. The ability of pulling the dial 77E and the outer sheath 2 in order to release the stent 6 is maximized when pulling starts and it decreases gradually. Since the bent groove 70Eg is provided with the first bent spot P1, the user can be prevented from continuously pulling the outer sheath 2 with the ability required when pulling starts. In addition, since the bent groove 70Eg is provided with the first bent spot P1, the user can temporarily stop the releasing operation in a state in which only a leading end portion of the stent 6 is released and can perform location adjustment of the stent 6.

A distance D2 from the leading end location S to the second bent spot P2 in the longitudinal direction A is approximately 50% of the distance D from the leading end location S to the proximal end location G in the longitudinal direction A. It is desirable that the center of the stent 6 be located at the center of stenosis when the stent 6 is being indwelled. Since the bent groove 70Eg is provided with the second bent spot P2, it is easy for the user to set the location of the stent 6 by temporarily stopping the stent operation of the stent 6 when the stent 6 is released approximately halfway.

A distance D3 from the leading end location S to the third bent spot P3 in the longitudinal direction A is approximately 80% of the distance D from the leading end location S to the proximal end location G in the longitudinal direction A. If the stent 6 is released 80% or more of the full length, it is generally difficult to recapture it. Since the bent groove 70Eg is provided with the third bent spot P3, it is easy for the user to recognize whether the stent 6 which is currently being released is in a state of being able to be recaptured.

A distance D4 from the proximal end location G to the fourth bent spot P4 in the longitudinal direction A is approximately 10 mm. When a treatment spot is the biliary tract, the stent 6 may be indwelled with a proximal end portion of the stent 6 protruding from the nipple by approximately 10 mm. Since the bent groove 70Eg is provided with the fourth bent spot P4, it is easy for the user to recognize whether the unreleased length of the stent 6 which is currently being released is approximately 10 mm.

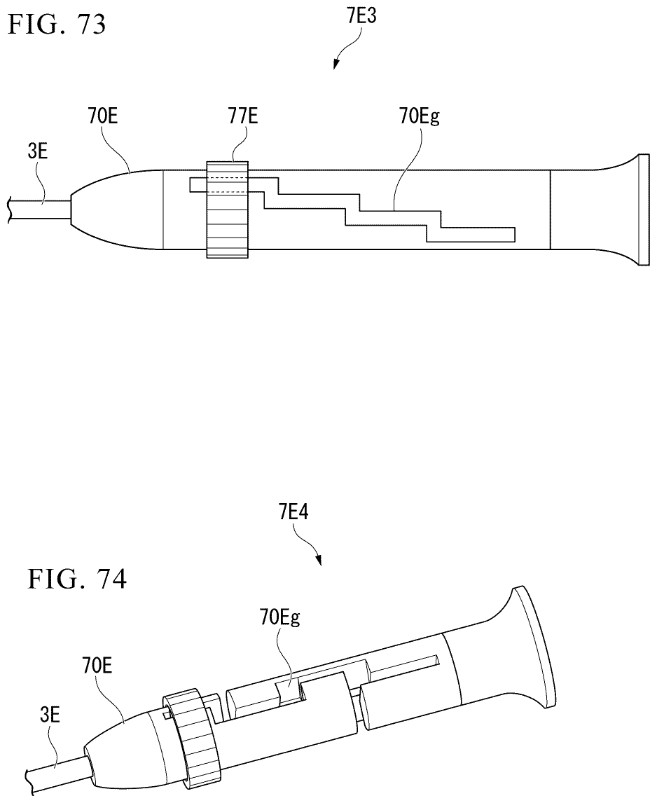

FIGS. 72 and 73 are diagrams representing an operation portion 7E3 that is another form of the present embodiment.

The bent spots P in the bent groove 70Eg formed in the operation portion main body 70E of the operation portion 7E3 bend in only one direction (clockwise, counterclockwise) of the circumferential direction C. The user can advance and retract the dial 77E by simply rotating the dial 77E in one direction of the circumferential direction C.

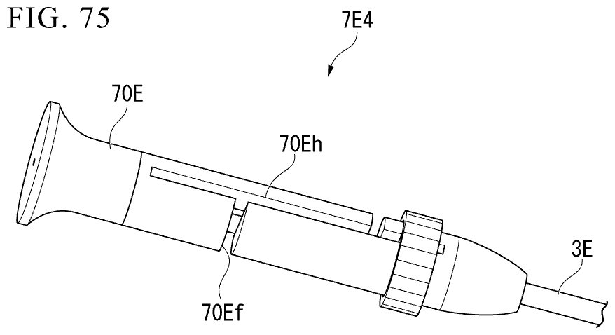

FIGS. 74 and 75 are diagrams representing an operation portion 7E4 that is another form of the present embodiment.

The operation portion main body 70E of the operation portion 7E4 has the bent groove 70Eg formed to have a zigzag shape, a straight groove 70Eh formed to have a straight shape, and a connection groove 70Ef connecting the bent groove 70Eg and the straight groove 70Eh. The user can move the dial 77E to the bent groove 70Eg or the straight groove 70Eh via the connection groove 70Ef. The user can use the bent groove 70Eg and the straight groove 70Eh differently in accordance with the circumstances of the procedure.

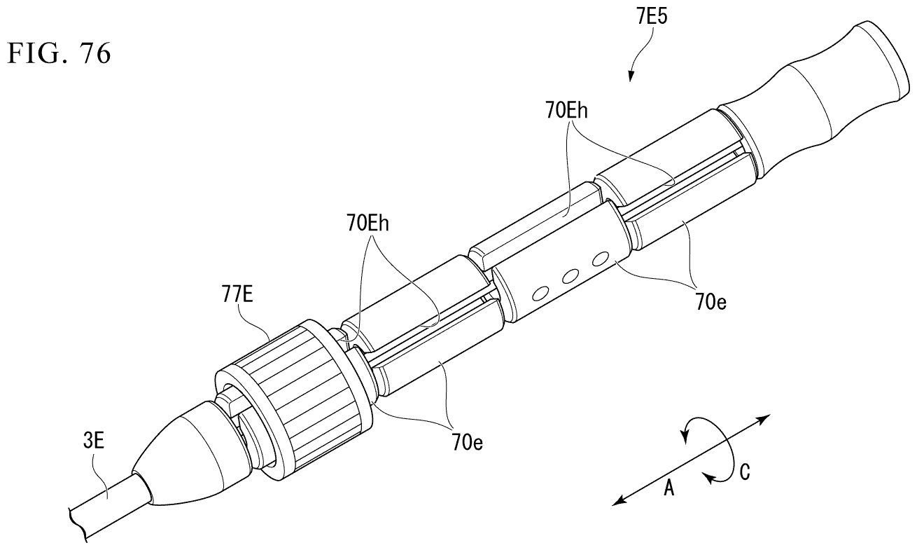

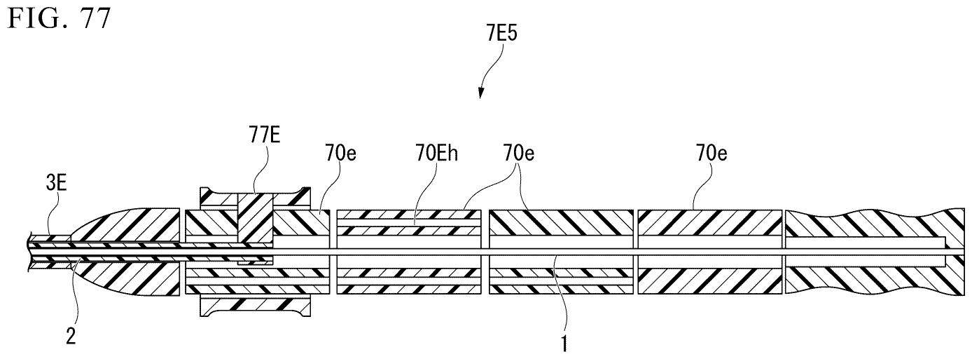

FIGS. 76 to 79 are diagrams representing an operation portion 7E5 that is another form of the present embodiment.

The operation portion 7E5 has a plurality of rotation members 70e, and the dial 77E. The plurality of rotation members 70e are arrayed in the longitudinal direction A. The rotation members 70e adjacent to each other in the longitudinal direction A are coupled such that they can relatively turn in the circumferential direction C.

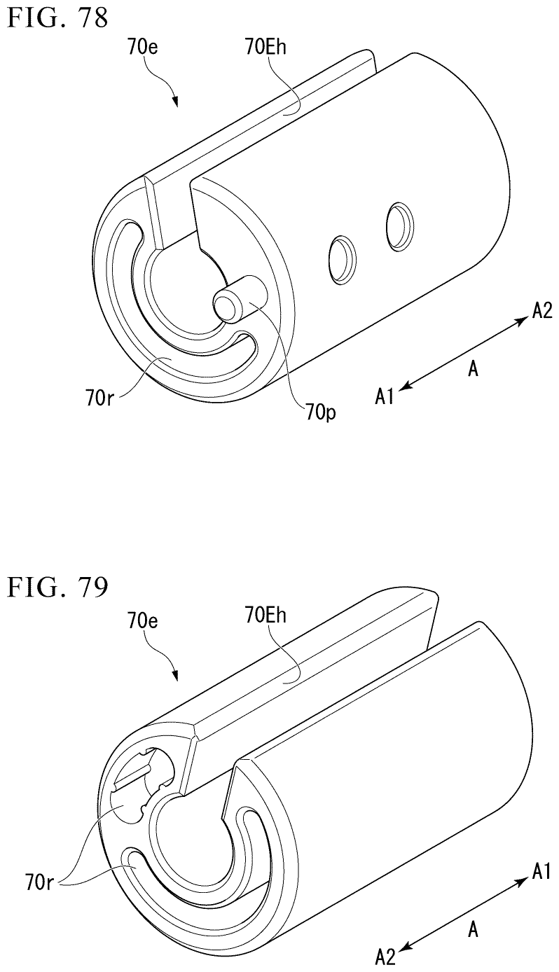

FIG. 78 is a perspective diagram of the rotation member 70e. FIG. 79 is another perspective diagram of the rotation member 70e viewed from a side opposite to that in FIG. 78. The straight groove 70Eh is formed in the rotation member 70e. The rotation member 70e has an engagement projection portion 70p and an engagement recessed portion 70r which engage with the adjacent rotation member 70e in the longitudinal direction A.

In the operation portion 7E5, the plurality of rotation members 70e can be disposed such that the straight grooves 70Eh of the plurality of rotation members 70e are not arranged in a straight line. In this case, if the user pulls the dial 77E to the proximal end side A2 along the straight grooves 70Eh of the rotation members 70e located closest to the leading end side A1, the dial 77E is caught by the rotation member 70e located second from the leading end side A1. The user can further pull the dial 77E to the proximal end side A2 by relatively rotating the rotation members 70e such that the straight groove 70Eh are arranged in a straight line. The user can release the stent 6 in stages by repeating the foregoing steps. The number of rotation members 70e and the lengths of the rotation members 70e in the longitudinal direction A are not limited.

In the operation portion 7E5, since it is possible to limit the amount of movement of the outer sheath 2 which can be moved by the user with one operation as described above, the user can be prevented from releasing the stent 6 by an unintended amount due to a human error. Moreover, by replacing the rotation members 70e, the user can freely select the location of the dial 77E to be caught and can temporarily stop the releasing operation reliably at a location desired to be confirmed depending on the case.

According to the stent delivery device 100E and the like of the present embodiment, the location of the inner sheath 1 is unlikely to deviate, and the location where the stent 6 is stored is unlikely to deviate from a target location. In addition, it is easy to control the amount of advancing/retracting movement of the outer sheath 2.

Hereinabove, the fifth embodiment of the present invention has been described in detail with reference to the drawings, but the specific constitutions are not limited to this embodiment, and design change and the like within a range not departing from the gist of the present invention are also included. In addition, the constituent elements described in the foregoing embodiment and the following modification examples can be suitably constituted in a combination.

### Sixth Embodiment

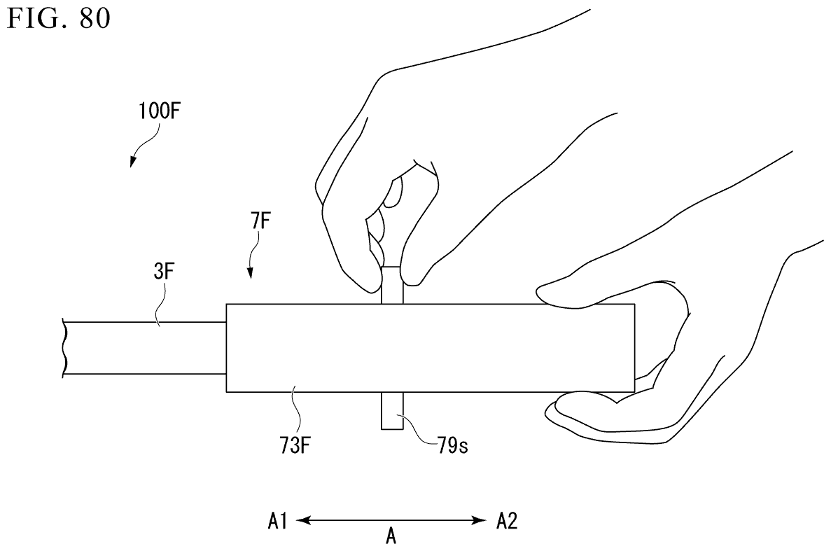

A sixth embodiment of the present invention will be described with reference to FIGS. 80 to 90. In the following description, the same reference signs are applied to common constituents which have already been described, and duplicate description will be omitted.

FIG. 80 is a diagram representing a stent delivery device 100F according to the present embodiment.

Similar to the stent delivery device 100 of the first embodiment, the stent delivery device 100F is inserted through the treatment tool channel 230 of the endoscope 200.

The stent delivery device 100F is formed to be thin and long in its entirety and includes the inner sheath (first sheath) 1, the outer sheath (second sheath) 2, an outermost layer sheath (third sheath) 3F, the tip 5, the stent 6, and an operation portion 7F. The stent delivery device 100F has a triple sheath structure in which the inner sheath 1, the outer sheath 2, and an outermost layer sheath 3F overlap.

The outermost layer sheath 3F has the outermost layer sheath main body 30. The proximal end of the outermost layer sheath main body 30 is attached to the operation portion 7F. When the stent delivery device 100F is used by being inserted into the treatment tool channel 230 of the endoscope 200, similar to the outermost layer sheath 3 of the second embodiment, a leading end of the outermost layer sheath 3F is disposed in a part which is inserted into the treatment tool channel 230.

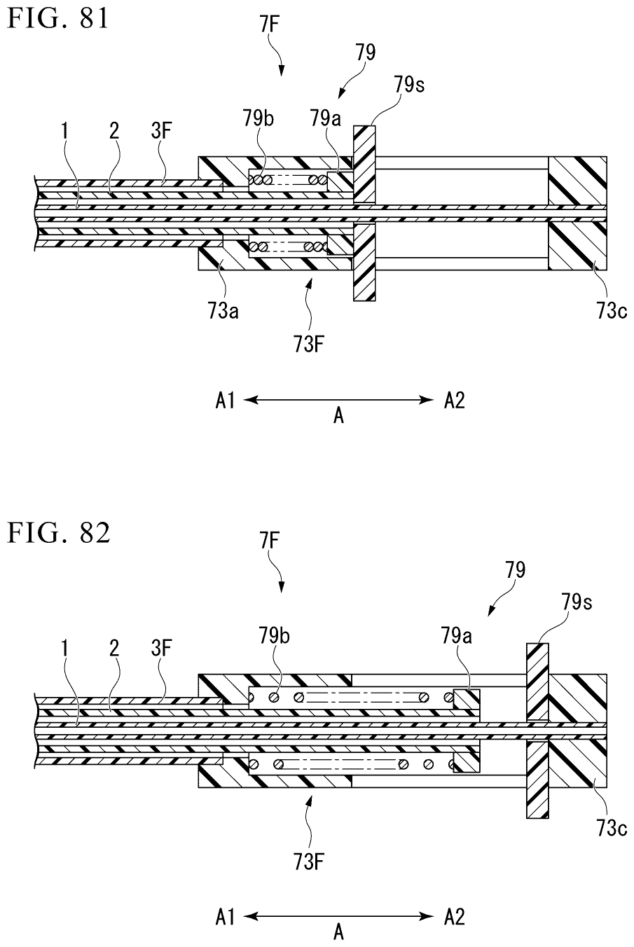

FIG. 81 is a cross-sectional diagram of the operation portion 7F.

The operation portion 7F has a box-shaped casing 73F and a release mechanism 79. The casing 73F surrounds the release mechanism 79.

The release mechanism 79 is a mechanism for releasing the stent 6 by pulling the outer sheath 2 with respect to the inner sheath 1. The release mechanism 79 has an outer joint member 79a, a compression spring 79b, and a stopper 79s.

The outer joint member 79a is disposed to be able to advance and retract in the longitudinal direction A in an internal space of the casing 73F. The outer joint member 79a is attached to the proximal end outer circumferential surface of the outer sheath 2.

The compression spring 79b is provided in the internal space of the casing 73F, in which a leading end is fixed to the casing leading end portion 73a of the casing 73F and a proximal end is fixed to the outer joint member 79a.

The stopper 79s is a stopper which can be attached and detached with respect to the casing 73F and fixes the location of the outer joint member 79a. The stopper 79s fixes the outer joint member 79a at a location closer to the leading end side such that the compression spring 79b is in a compressed state.

FIG. 82 is a diagram representing the release mechanism 79 after the stent 6 has been released.

The user unfixes the stopper 79s from the casing 73F. Accordingly, the compression spring 79b extends in the longitudinal direction A and retracts the outer joint member 79a. When the outer joint member 79a retracts, the outer sheath 2 is pulled, and the stent 6 is released.

Since there is no need for the user to directly perform the operation of releasing the stent 6, the stent 6 can be released with no human error, and the location where the stent 6 is stored is unlikely to deviate from a target location.

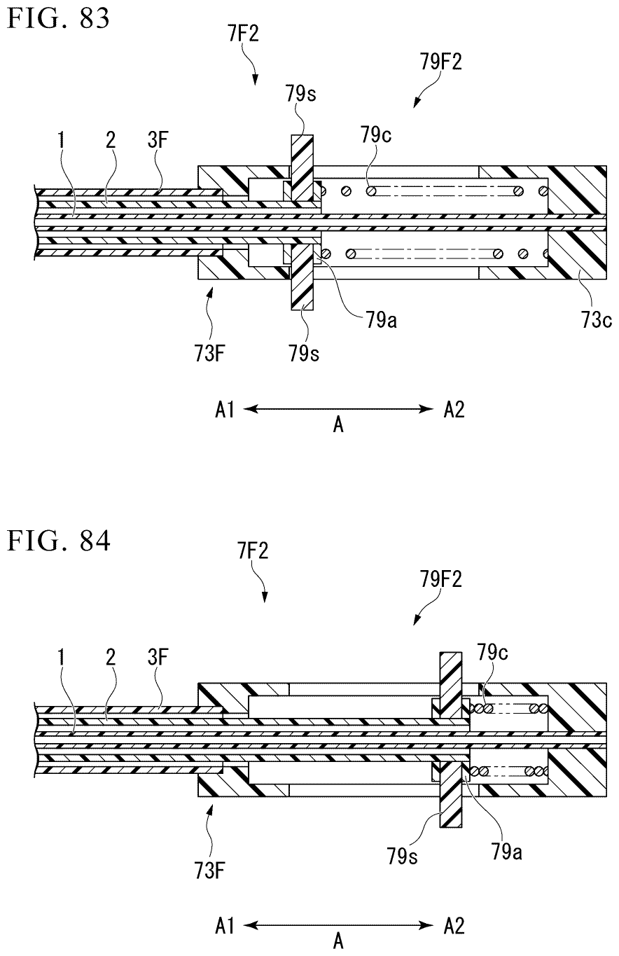

FIGS. 83 and 84 are diagrams representing an operation portion 7F2 that is another form of the present embodiment.

The operation portion 7F2 has the box-shaped casing 73F and a release mechanism 79F2.

The release mechanism 79F2 is a mechanism for releasing the stent 6 by pulling the outer sheath 2. The release mechanism 79F2 has the outer joint member 79a, a tension spring 79c, and the stopper 79s.

The tension spring 79c is provided in the internal space of the casing 73F, in which a leading end is fixed to the outer joint member 79a and a proximal end is fixed to the casing proximal end portion 73c of the casing 73F.

The stopper 79s is a stopper which can be attached and detached with respect to the casing 73F and fixes the location of the outer joint member 79a. The stopper 79s fixes the outer joint member 79a at a location closer to the leading end side such that the tension spring 79c is in a tensed state.

The user unfixes the stopper 79s from the casing 73F. Accordingly, the tension spring 79c shrinks in the longitudinal direction A and retracts the outer joint member 79a. When the outer joint member 79a retracts, the outer sheath 2 is pulled, and the stent 6 is released.

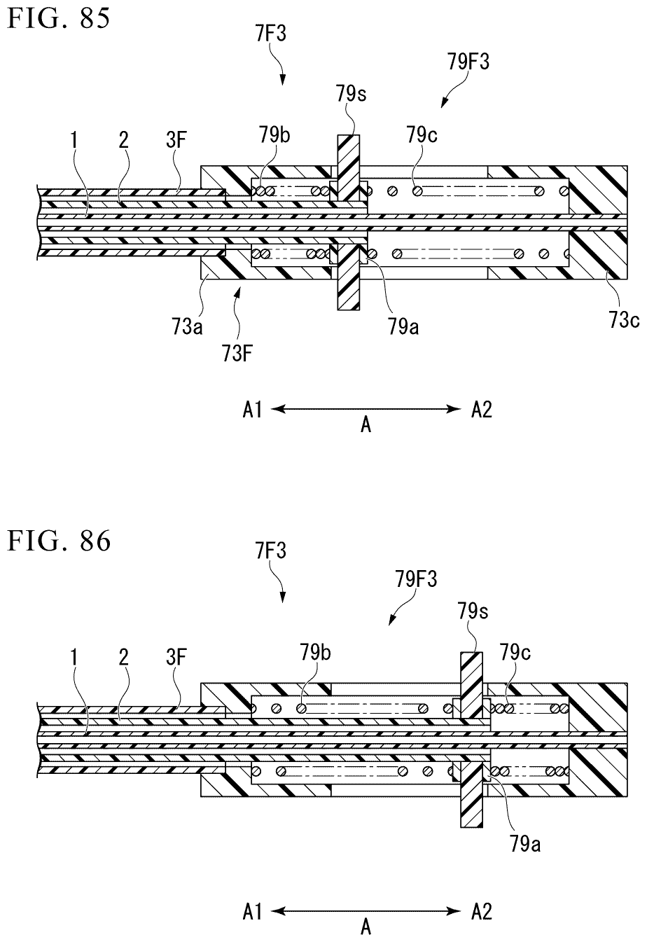

FIGS. 85 and 86 are diagrams representing an operation portion 7F3 that is another form of the present embodiment.

The operation portion 7F3 has the box-shaped casing 73F and a release mechanism 79F3.

The release mechanism 79F3 is a mechanism for releasing the stent 6 by pulling the outer sheath 2. The release mechanism 79F3 has the outer joint member 79a, the compression spring 79b, the tension spring 79c, and the stopper 79s.

The user unfixes the stopper 79s from the casing 73F. Accordingly, the compression spring 79b extends in the longitudinal direction A and retracts the outer joint member 79a. In addition, the tension spring 79c shrinks in the longitudinal direction A and retracts the outer joint member 79a. When the outer joint member 79a retracts, the outer sheath 2 is pulled, and the stent 6 is released.

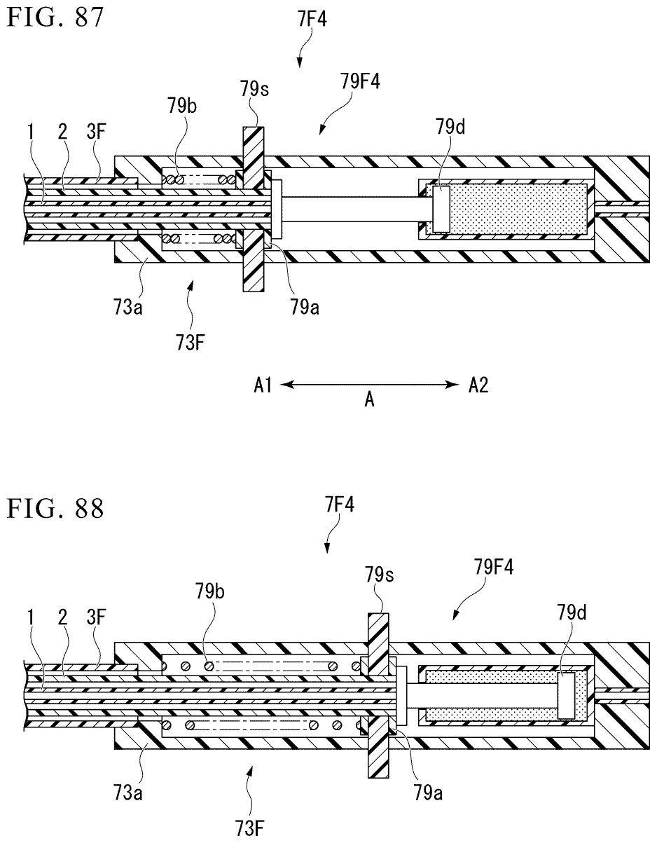

FIGS. 87 and 88 are diagrams representing an operation portion 7F4 that is another form of the present embodiment.

The operation portion 7F4 has the box-shaped casing 73F and a release mechanism 79F4.

The release mechanism 79F4 is a mechanism for releasing the stent 6 by pulling the outer sheath 2. The release mechanism 79F4 has the outer joint member 79a, the compression spring 79b, a damper (damping member) 79d, and the stopper 79s.

The user unfixes the stopper 79s from the casing 73F. Accordingly, the compression spring 79b extends in the longitudinal direction A and retracts the outer joint member 79a. The outer joint member 79a slowly retracts due to the damper 79d. When the outer joint member 79a retracts, the outer sheath 2 is pulled, and the stent 6 is released. The damper 79d can prevent the stent 6 from being vigorously released.

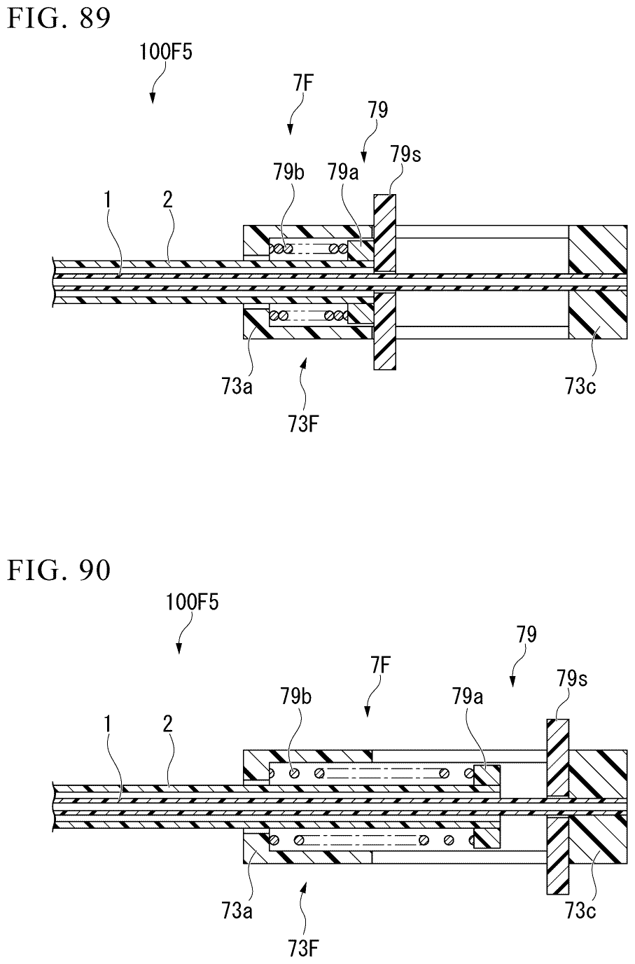

FIGS. 89 and 90 are diagrams representing a stent delivery device 100F5 that is another form of the present embodiment. The stent delivery device 100F5 is formed to be thin and long in its entirety and includes the inner sheath (first sheath) 1, the outer sheath (second sheath) 2, the tip 5, the stent 6, and the operation portion 7F. The stent delivery device 100F5 has a double sheath structure which does not have the outermost layer sheath 3E. The operation portion 7F can also be attached to a double sheath (the inner sheath 1 and the outer sheath 2) which does not have the outermost layer sheath 3E.

According to the stent delivery device 100F and the like of the present embodiment, since there is no need for the user to directly perform the operation of releasing the stent 6, there is no human error, the location of the inner sheath 1 is unlikely to deviate, and the location where the stent 6 is stored is unlikely to deviate from a target location.

Hereinabove, the sixth embodiment of the present invention has been described in detail with reference to the drawings, but the specific constitutions are not limited to this embodiment, and design change and the like within a range not departing from the gist of the present invention are also included. In addition, the constituent elements described in the foregoing embodiment and the following modification examples can be suitably constituted in a combination.

### Seventh Embodiment

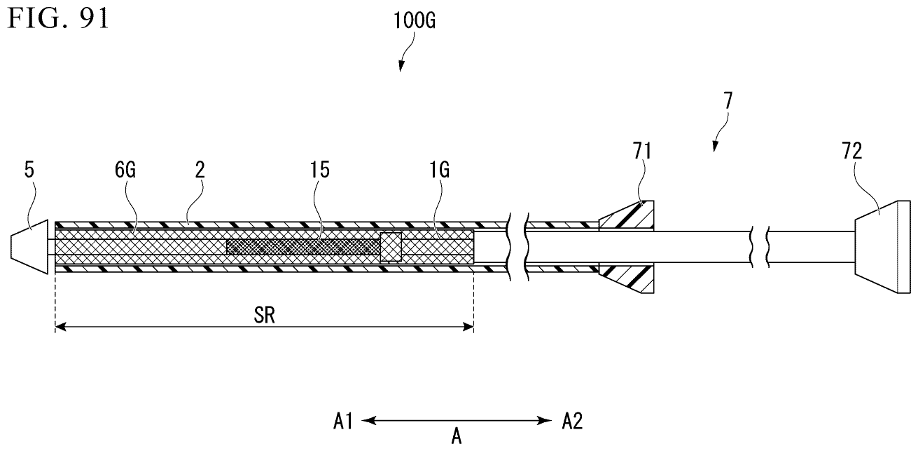

A seventh embodiment of the present invention will be described with reference to FIGS. 91 to 97. In the following description, the same reference signs are applied to common constituents which have already been described, and duplicate description will be omitted.

FIG. 91 is a diagram representing a stent delivery device 100G according to the present embodiment.

Similar to the stent delivery device 100 of the first embodiment, the stent delivery device 100G is inserted through the treatment tool channel 230 of the endoscope 200.

The stent delivery device 100G is formed to be thin and long in its entirety and includes an inner sheath (first sheath) 1G, the outer sheath (second sheath) 2, the tip 5, a stent 6G, and the operation portion 7.

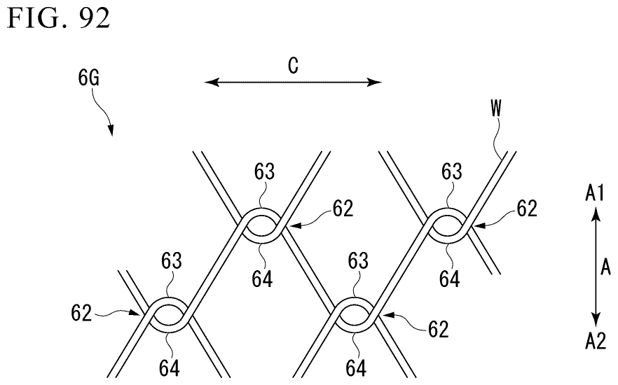

FIG. 92 is a diagram representing the stent 6G.

The stent 6G is formed by weaving wires W and has a cylindrical shape. The stent 6G is formed to have a circular tube shape having a mesh pattern on its circumferential surface using the wires W which extend in an inclined manner in the circumferential direction C while repeatedly bending. The stent 6G has a plurality of entangled portions 62.

The entangled portions (intertwined portions) 62 are formed by mountain-type bent portions 63 and valley-type bent portions 64 intersecting each other. The mountain-type bent portions (mountains) 63 are projection portions where the wires W inclined and extending in the circumferential direction C are folded back and bend in a longitudinal axis direction A and projecting to a first direction A1 side. The valley-type bent portions (valleys) 64 are projection portions where the wires W inclined and extending in the circumferential direction C are folded back and bend in the longitudinal direction A and projecting to a second direction A2 side (recessed portions which are recessed on the first direction A1 side). In the entangled portions 62, the mountain-type bent portions 63 and the valley-type bent portions 64 intersect in a hook shape so that the mountain-type bent portions 63 and the valley-type bent portions 64 are coupled in a manner of being inseparable but relatively movable.

The stent 6G is a so-called fence netting stent, which has a small repulsive force against bending (axial force) and is likely to bend along the shape of an organ.

The inner sheath (first sheath) 1G differs from the inner sheath 1 of the first embodiment in further having a high-friction member 15 on the outer circumferential surface of the inner sheath 1G.

The high-friction member 15 is a member having a higher frictional coefficient than other parts on the outer circumferential surface of the inner sheath 1G. The high-friction member 15 is provided in the stent storage region SR and comes into contact with the stored stent 6G. The high-friction member 15 is not provided in a leading end portion of the stent storage region SR. Specifically, the high-friction member 15 is not disposed at least in the region in which the cell closest to the leading end side (a space surrounded by the wires W) in the stent 6G stored in the stent storage region SR is disposed.

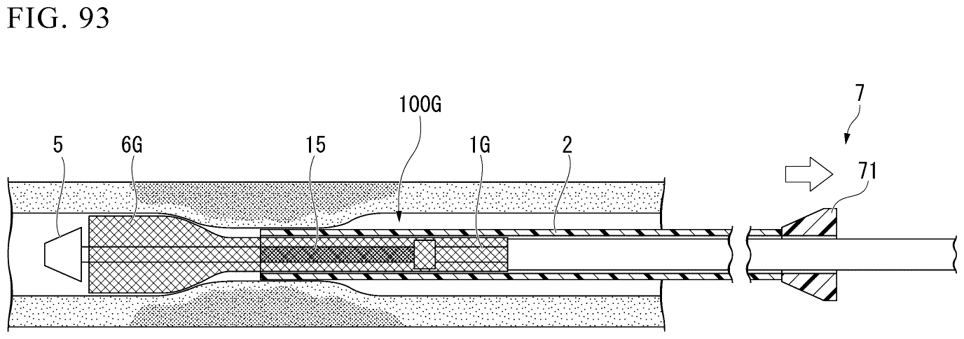

FIG. 93 is another diagram representing the stent 6G being released.

The user retracts the outer operation handle 71 with respect to the inner operation handle 72 by operating the operation portion 7. The outer sheath 2 retracts with respect to the inner sheath 1G. As a result, the stent 6G is gradually exposed from the leading end side and expands.

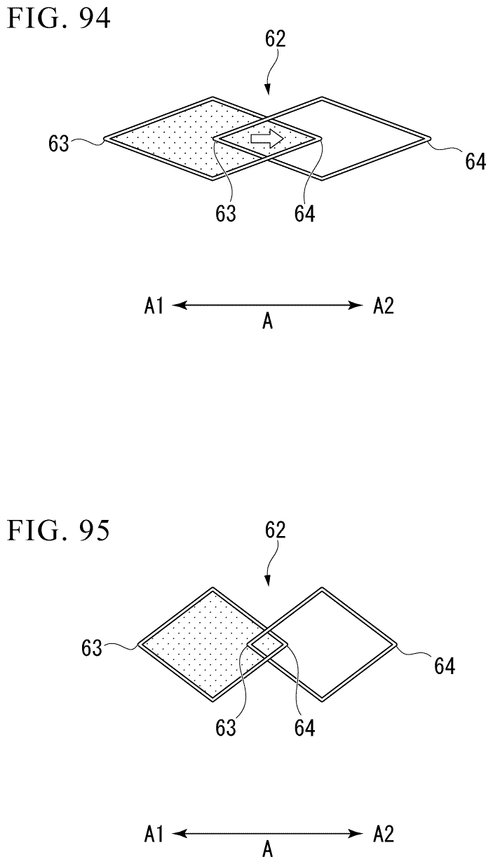

FIG. 94 is an explanatory diagram of axial collapse of the stent 6G.

Since the stent 6G stored in the stent storage region SR tends to expand in the radial direction R due to a self-expanding force, it comes into contact with an inner circumferential surface of the outer sheath 2. For this reason, if the outer sheath 2 retracts, the cell which comes into contact with the outer sheath 2 moves to the proximal end side so that the stent 6G collapses in the axial direction and is shortened in the longitudinal direction A. This phenomenon will be referred to as “axial collapse”. The relative location of an axially collapsed cell is likely to deviate from those of adjacent cells, and the axially collapsed cell is likely to be caught by the outer sheath 2 at the time of recapturing.

The leading end portion of the stent storage region SR is not provided with the high-friction member 15. For this reason, a leading end portion of the stent 6G stored in the leading end portion of the stent storage region SR is likely to axially collapse at the time of releasing. Meanwhile, the part of the stent 6G stored in the part where the high-friction member 15 in the stent storage region SR is provided is unlikely to axially collapse at the time of releasing. This is because a frictional force between the stent 6G and the inner sheath 1G is significant.

FIG. 95 is a diagram representing the leading end portion of the stent 6G being released.

The leading end portion of the released stent 6G expands in the radial direction R due to a self-expanding force. As a result, it extends in the circumferential direction C and is shortened in the longitudinal direction A. As a result, axial collapse is resolved as represented in FIG. 95.

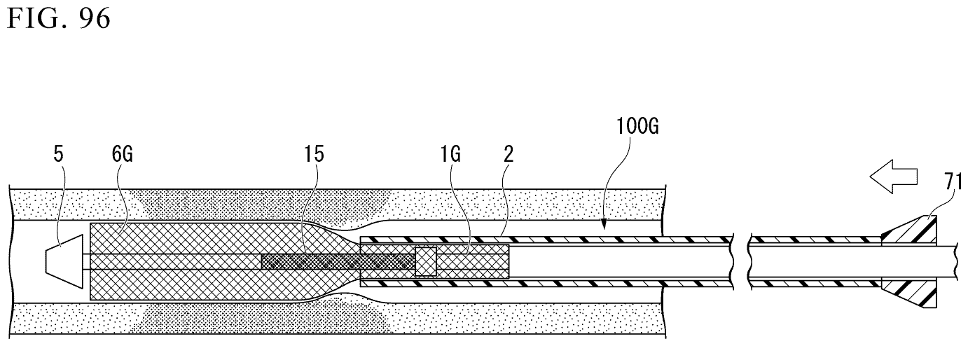

FIG. 96 is a diagram representing the leading end portion of the stent 6G being recaptured.

Since axial collapse of the leading end portion of the released stent 6G is resolved, the wires W can be recaptured without being caught by the outer sheath 2.

In order to prevent axial collapse (collapse in the axial direction) of the stent 6G when the stent 6G is released, it is desirable that the high-friction member 15 be provided in the entire area of the stent storage region SR. However, if the high-friction member 15 is provided in the entire area of the stent storage region SR, a frictional force generated between the stent 6G and the inner sheath 1G increases, and therefore the ability required to retract the outer sheath 2 in order to release the stent 6G (which will hereinafter be referred to as “release ability”) increases. That is, there is a trade-off relationship between the range in which the high-friction member 15 is provided and the release ability.

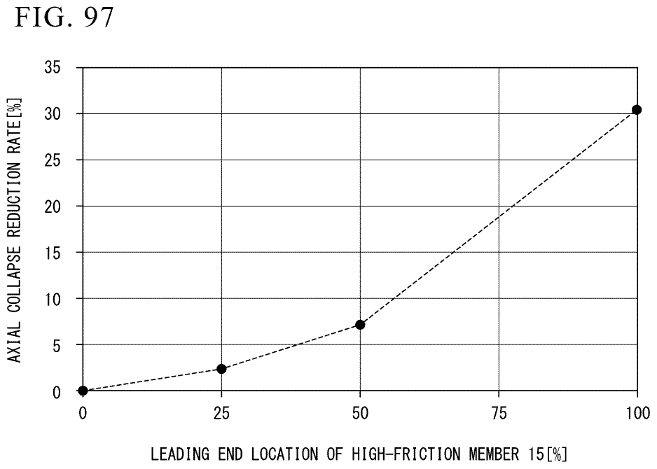

FIG. 97 is a graph representing a relationship between a leading end location of the high-friction member 15 and an axial collapse reduction rate.

The leading end location of the high-friction member 15 indicates the ratio of the length from a leading end of the stent storage region SR to the leading end location of the high-friction member 15 to the full length of the stent storage region SR. The axial collapse reduction rate indicates the ratio of the length of axial collapse occurring when the stent 6G is released to the full length of the stent 6G. When the leading end location of the high-friction member 15 is within a range of 50% to 100% of the full length of the stent storage region SR from the leading end of the stent storage region SR, the axial collapse reduction rate is higher than those in other ranges, and the rate of increase (inclination) in the axial collapse reduction rate increases. For this reason, it is desirable that the leading end location of the high-friction member 15 be disposed within 50% of the full length of the stent storage region SR from the leading end of the stent storage region SR.

According to the stent delivery device 100G of the present embodiment, the stent 6G having a small repulsive force against bending (axial force) is likely to be recaptured, and the stent 6G has small release ability. Even if a stent stored in the stent delivery device 100G is a different kind of stent having a larger repulsive force against bending (axial force) than the stent 6G, it is easy for the stent delivery device 100G to recapture the stent, and the stent has small release ability.

Hereinabove, the seventh embodiment of the present invention has been described in detail with reference to the drawings, but the specific constitutions are not limited to this embodiment, and design change and the like within a range not departing from the gist of the present invention are also included. In addition, the constituent elements described in the foregoing embodiment and the following modification examples can be suitably constituted in a combination.

### Modification Example 7-1

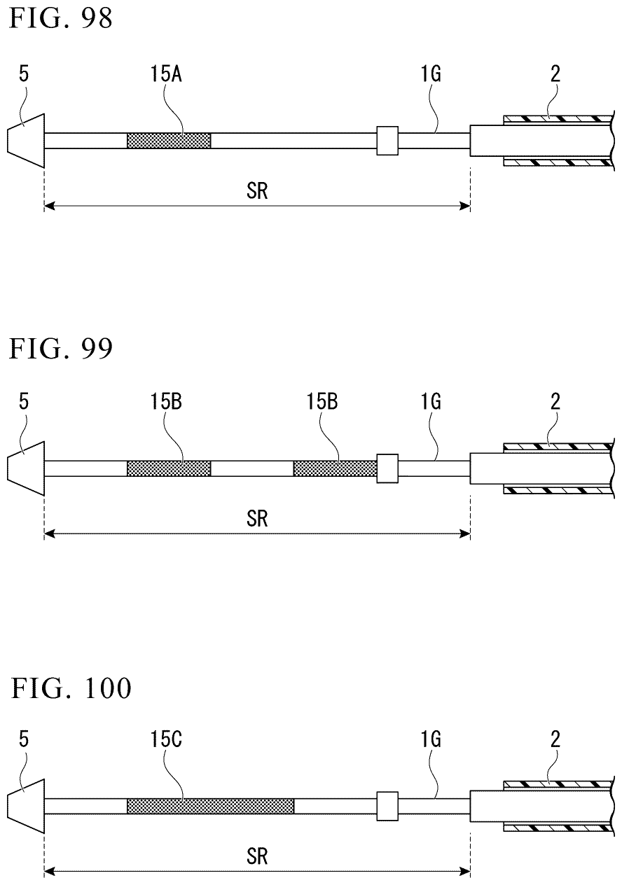

FIGS. 98 to 100 are diagrams respectively representing high-friction members 15A, 15B, and 15C that are modification examples of the high-friction member 15. None of the modification examples of the high-friction member 15 is provided in the leading end portion of the stent storage region SR. The high-friction member 15A is not provided in the rear half part of the stent storage region SR. The high-friction member 15B is provided in a manner of being divided into two in the longitudinal direction A. The high-friction member 15C is not provided in a proximal end portion of the stent storage region SR.

### Modification Example 7-1

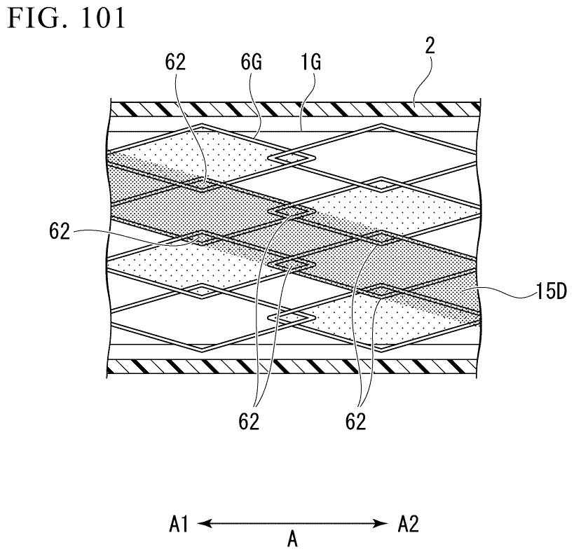

FIG. 101 is a diagram representing a high-friction member 15D that is another modification example of the high-friction member 15. The high-friction member 15D is provided in only a part on the outer circumferential surface of the inner sheath 1G in the circumferential direction C. From the viewpoint of reducing occurrence of axial collapse, it is desirable that the high-friction member 15D be disposed so as to come into contact with the entangled portions 62 of the stored stent 6G as much as possible.

### Eighth Embodiment)

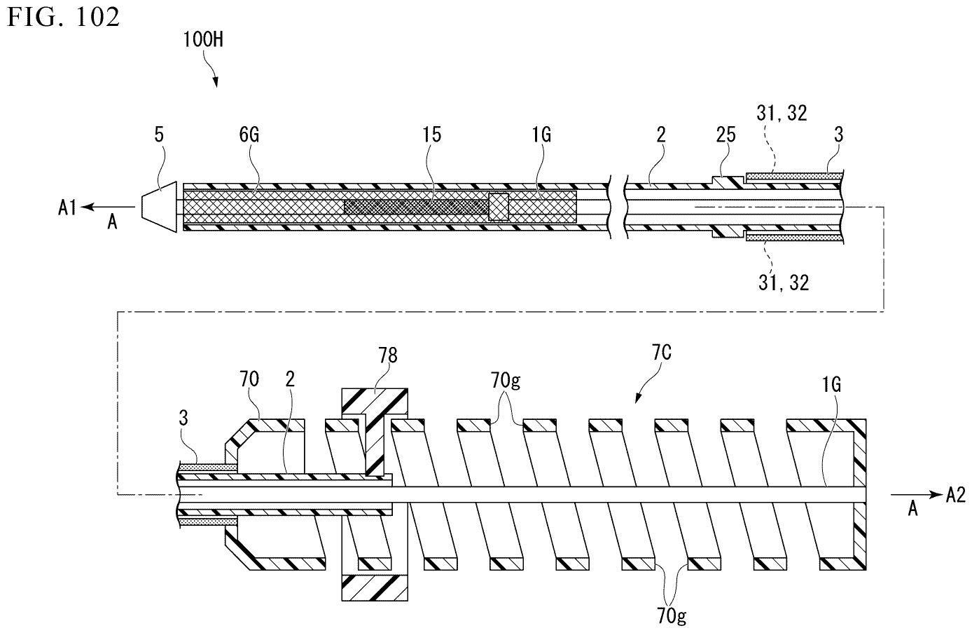

An eighth embodiment of the present invention will be described with reference to FIG. 102. The eighth embodiment is an embodiment in which the first to seventh embodiments described above are combined. In the following description, the same reference signs are applied to common constituents which have already been described, and duplicate description will be omitted.

FIG. 102 is a diagram representing a stent delivery device 100H that is an example of a combination of the embodiments. The stent delivery device 100H represented in FIG. 102 as an example is a combination of a part of the second embodiment, a part of the third embodiment, and a part of the seventh embodiment. The stent delivery device 100H is formed to be thin and long in its entirety and includes the inner sheath (first sheath) 1G, the outer sheath (second sheath) 2, the outermost layer sheath (third sheath) 3, the tip 5, the stent 6G, and the operation portion 7C. The stent delivery device 100H also has the effects which have been described in the description of the embodiments.

According to the stent delivery device 100H of the present embodiment, releasing the stent 6G is facilitated.

Combinations of the embodiments are not limited to the stent delivery device 100H represented in FIG. 102 as an example. Combinations of the foregoing embodiments are arbitrary.

Hereinabove, the eighth embodiment of the present invention has been described in detail with reference to the drawings, but the specific constitutions are not limited to this embodiment, and design change and the like within a range not departing from the gist of the present invention are also included. In addition, the constituent elements described in the foregoing embodiment and the following modification examples can be suitably constituted in a combination.

The present invention can be applied to a stent delivery device and the like.

What is claimed is:

1. A stent delivery device comprising:
an outer sheath;
an inner sheath which is inserted through an inward side of the outer sheath;
an outermost layer sheath through which the outer sheath is inserted on the inward side;
an operation portion which has a casing coupling the inner sheath and the outermost layer sheath and is capable of operating the outer sheath and the inner sheath; and
a fixing mechanism which fixes a channel of an endoscope and the outermost layer sheath.

2. The stent delivery device according to claim 1,
wherein the fixing mechanism varies between a constitution separated from the channel of the endoscope and a constitution in contact with the channel of the endoscope.

3. The stent delivery device according to claim 1,
wherein the fixing mechanism is a stretchable portion provided in the outermost layer sheath, and
the stretchable portion varies between a stretched constitution and a folded constitution in response to movement of the outer sheath.

4. The stent delivery device according to claim 1,
wherein the fixing mechanism is a stretchable portion provided in the outermost layer sheath, and
the stretchable portion is folded upon contact with the outer sheath retracting.

5. The stent delivery device according to claim 4,
wherein the operation portion has an elastic member causing the outer sheath that has retracted to advance.

6. The stent delivery device according to claim 1,
wherein the fixing mechanism is a stretchable portion provided in the outermost layer sheath, and
the stretchable portion is pulled by the outer sheath retracting and is folded.

7. The stent delivery device according to claim 1 further comprising:
a self-expanding sheath which is disposed between the outer sheath and the outermost layer sheath,
wherein the self-expanding sheath has a self-expanding portion having a self-expanding force at a leading end, and
the fixing mechanism is the self-expanding portion of the self-expanding sheath protruding from a leading end of the outermost layer sheath.

8. The stent delivery device according to claim 1 further comprising:
a cover which is attached to be able to advance and retract in conjunction with the outer sheath on an outward side of the outermost layer sheath,
wherein the outermost layer sheath has a self-expanding portion having a self-expanding force at a leading end, and
the fixing mechanism is the self-expanding portion of the outermost layer sheath protruding from a leading end of the cover.

9. The stent delivery device according to claim 1,
wherein the fixing mechanism is an engagement member fixing the endoscope and the outermost layer sheath.

10. The stent delivery device according to claim 9,
wherein the engagement member is provided in the outermost layer sheath and has a rubber material.

11. The stent delivery device according to claim 9,
wherein the engagement member is a pinch member having a pinch shape.

12. The stent delivery device according to claim 9,
wherein the engagement member is a guide wire locking device.
